(19) United States
(12) Patent Application Publication
(10) Pub. No.: US 2024/0398424 A1
(43) Pub. Date: Dec. 5, 2024

(54) CONCENTRIC TUBE DRILLING ROBOT DEVICE, SYSTEM AND METHOD

(71) Applicants: Jordan AMADIO, Austin, TX (US); Board of Regents, The University of Texas System, Austin, TX (US)

(72) Inventors: Farshid Alambeigi, Austin, TX (US); Susheela Sharma, Austin, TX (US); Jordan Amadio, Austin, TX (US)

(21) Appl. No.: 18/702,407

(22) PCT Filed: Oct. 28, 2022
(86) PCT No.: PCT/US2022/078881

§ 371 Date: Apr. 18, 2024

Related U.S. Application Data
(60) Provisional application No. 63/272,895, filed on Oct. 28, 2021.

(51) Int. Cl. A61B 17/16; A61B 17/00; A61B 34/10; A61B 34/30; B25J 9/16

(52) U.S. Cl. CPC A61B 17/1631; A61B 17/1628; A61B 17/1671; A61B 34/10; A61B 34/30; B25J 9/1633; A61B 2017/00867; A61B 17/1668; A61B 17/1675; A61B 2034/105; A61B 2034/107

(57) ABSTRACT

A concentric tube drilling robot device comprises an outer tube including a proximal end, a distal end, and a concentric through hole centered on a longitudinal axis extending from the proximal end to the distal end, at least one inner tube nested within the outer tube including a proximal end, a distal end, and a concentric through hole centered on the longitudinal axis, wherein the at least one nested inner tube is concentric with the outer tube, and a flexible drive shaft including a proximal end, a distal end and a tool tip positioned at the distal end, wherein the flexible drive shaft extends through the concentric through hole of the at least one inner tube, and is configured to provide a rotational torque to the tool tip. A drilling robot system and drilling method are also disclosed.

### CROSS-REFERENCE TO RELATED APPLICATIONS

This application claims priority to U.S. provisional application No. 63/272,895 filed on Oct. 28, 2021, incorporated herein by reference in its entirety.

### STATEMENT REGARDING FEDERALLY SPONSORED RESEARCH OR DEVELOPMENT

This invention was made with government support under R21 EB030796 awarded by the National Institutes of Health. The government has certain rights in the invention.

### BACKGROUND OF THE INVENTION

Bone is the most common site of metastatic disease after lung and liver and one of the most common causes of chronic pain among cancer patients (see C. Pusceddu et al., “Treatment of bone metastases with microwave thermal ablation,” Journal of Vascular and Interventional Radiology, vol. 24, no. 2, pp. 229-233, 2013) (see N. C. Hatrick et al., “The surgical treatment of metastatic disease of the spine,” Radiotherapy and Oncology, vol. 56, no. 3, pp. 335-339, 2000) (see W. B. Jacobs and R. G. Perrin, “Evaluation and treatment of spinal metastases: an overview,” Neurosurgical focus, vol. 11, no. 6, pp. 1-11, 2001) (see G. Maccauro et al., “Physiopathology of spine metastasis,” International journal of surgical oncology, vol. 2011, 2011). Each year, 400,000 people in the US alone suffer from bone metastases, which includes two-thirds of patients with metastatic disease. The most frequent site of bone metastasis is seen in the spine and particularly vertebrae, which includes 50% of all bone metastatic disease (FIG. 9P) (see D. Choi et al., “Review of metastatic spine tumour classification and indications for surgery: the consensus statement of the global spine tumour study group,” European spine journal, vol. 19, no. 2, pp. 215-222, 2010) (see T. J. Greenwood et al., “Combined ablation and radiation therapy of spinal metastases: a novel multimodality treatment approach,” Pain physician, vol. 18, no. 6, pp. 573-581, 2015) (see M. P. Steinmetz et al., “Management of metastatic tumors of the spine: strategies and operative indications,” Neurosurgical Focus, vol. 11, no. 6, pp. 1-6, 2001). Traditional therapeutic methods for spinal tumors involve chemotherapy and possibly radiation, pain management, etc. However, many patients have a minimal or only brief response to these therapies and onset of pain relief can take months (see P. R. Anchala et al., “Treatment of metastatic spinal lesions with a navigational bipolar radiofrequency ablation device: a multicenter retrospective study,” Pain physician, vol. 17, no. 4, 2014). Additionally, invasive surgical procedures depend on the nature and location of the tumor and are usually not warranted in these patient populations due to significant blood loss, post-op pain, wound healing issues, and coexisting health problems see C. Pusceddu et al., “Treatment of bone metastases with microwave thermal ablation,” Journal of Vascular and Interventional Radiology, vol. 24, no. 2, pp. 229-233, 2013) (see W. Sze et al., “Palliation of metastatic bone pain: single fraction versus multifraction radiotherapy-a systematic review of randomised trials,” Clinical Oncology, vol. 15, no. 6, pp. 345-352, 2003).

Despite invasive surgical methods, minimally invasive (MI) procedures such as thermal ablation, vertebroplasty, and kyphoplasty are safe and effective treatments of painful spinal metastatic lesions (see M. P. Steinmetz et al., “Management of metastatic tumors of the spine: strategies and operative indications,” Neurosurgical Focus, vol. 11, no. 6, pp. 1-6, 2001). In these manual image guided procedures, typically, a surgeon advances a rigid instrument through the skin and vertebra on a patient's back under X-ray guidance to confirm that it has entered the lesion area in vertebra. Then, to stabilize the typically fractured or degraded bone in vertebra, a rigid pedicle screw is utilized and/or bone cement, using a rigid syringe, is injected into the fractured vertebra for fixation (see S. Becker et al., “Assessment of different screw augmentation techniques and screw designs in osteoporotic spines,” European Spine Journal, vol. 17, no. 11, pp. 1462-1469, 2008). However, due to the rigidity of the utilized instruments and complex and sensitive anatomy of spine surrounded by nerves, these procedures typically suffer from the lack of enough accessibility to the tumor lesion and therefore cannot completely remove/treat the tumor and in some cases may even increase the risk of tumor spread to blood vessels (see D. A. Nussbaum et al., “A review of complications associated with vertebroplasty and kyphoplasty as reported to the food and drug administration medical device related web site,” Journal of Vascular and Interventional Radiology, vol. 15, no. 11, pp. 1185-1192, 2004).

To address the aforementioned limitation of existing rigid instruments, surgeons can use novel flexible robotic systems to minimally invasively navigate to harder to reach regions within the vertebral body by drilling in curved trajectories and reach the tumor area (see F. Alambeigi et al., “A curved-drilling approach in core decompression of the femoral head osteonecrosis using a continuum manipulator,” IEEE Robotics and Automation Letters, vol. 2, no. 3, pp. 1480-1487, 2017) (see F. Alambeigi et al., “Inroads toward robot-assisted internal fixation of bone fractures using a bendable medical screw and the curved drilling technique,” in 2018 7th IEEE International Conference on Biomedical Robotics and Biomechatronics (Biorob). IEEE, 2018, pp. 595-600) (see F. Alambeigi et al., “On the use of a continuum manipulator and a bendable medical screw for minimally invasive interventions in orthopedic surgery,” IEEE transactions on medical robotics and bionics, vol. 1, no. 1, pp. 14-21, 2019.). After the drilling procedure, using the robotic system, different treatment procedures can then be delivered locally and precisely to the tumor area to deliver a high dose radiation for brachytherapy (see S. L. Zuckerman et al., “Brachytherapy in spinal tumors: A systematic review,” World Neurosurgery, vol. 118, pp. e235-e244, 2018) and ablation procedures (see C. L. Brace, “Radiofrequency and microwave ablation of the liver, lung, kidney, and bone: what are the differences?” Current problems in diagnostic radiology, vol. 38, no. 3, pp. 135-143, 2009), or the robot can be used to completely excise the tumor area (see L. B. Kratchman et al., “Design of a bone-attached parallel robot for percutaneous cochlear implantation,” IEEE Transactions on Biomedical Engineering, vol. 58, no. 10, pp. 2904-2910, 2011) (see T. Yang et al., “6-surgical positioning, navigation, important surgical tools, craniotomy, and closure of cranial and spinal wounds,” in Principles of Neurological Surgery (Fourth Edition), fourth edition ed., R. G. Ellenbogen, L. N. Sekhar, N. D. Kitchen, and H. B. da Silva, Eds. Philadelphia: Elsevier, 2018, pp. 103-115.e1). Nevertheless, these applications require a flexible yet structurally strong flexible robot that is able to safely drill through the bone and access these difficult to reach areas without buckling and structural failure. Ensuring this balance between structural stiffness and flexible dexterity is the essential challenge in designing steerable drilling robotic systems [14], [19], [20].

Osteoporosis is a serious public health concern, described as a generalized decrease in bone mineral density (BMD) by more than 2.5 standard deviations below the healthy population mean (see O. of the Surgeon General (US et al., “Bone health and osteoporosis: a report of the surgeon general,” 2004) (see J. A. Kanis et al., “The diagnosis of osteoporosis,” Journal of bone and mineral research, vol. 9, no. 8, pp. 1137-1141, 1994). Osteoporosis is responsible for an estimated 2 million broken bones per year in the United States (see. C. Wright et al., “The recent prevalence of osteoporosis and low bone mass in the united states based on bone mineral density at the femoral neck or lumbar spine,” Journal of Bone and Mineral Research, vol. 29, no. 11, pp. 2520-2526, 2014). Of these osteoporotic fractures, vertebral compression fractures are the most common type (about 47%) with more than 1.4 million global incidence in men and women over age 50 (see O. Johnell and J. Kanis, “An estimate of the worldwide prevalence and disability associated with osteoporotic fractures,” Osteoporosis international, vol. 17, no. 12, pp. 1726-1733, 2006). In fractures too unstable for nonsurgical methods (e.g., medications and orthotic bracing), patients may require a spinal fusion surgery. In this intervention, with the goal of internal fixation of the spine, two (or more) vertebrae are locked together using rigid pedicle screws or cables through facet joints so that they can fuse into one solid bone, eliminating painful motion and restoring stability to the spine. This same procedure is also commonly performed in elective fusion operations for degenerative spine disease, which predominantly affects older adults and frequently co-occurs with osteoporosis (see C. M. Klotzbuecheret al., “Patients with prior fractures have an increased risk of future fractures: a summary of the literature and statistical synthesis,” Journal of bone and mineral research, vol. 15, no. 4, pp. 721-739, 2000).

Despite the well-established benefits of a fusion procedure, for an osteoporotic vertebral body, stability of fusion with rigid screws or cables through facet joints is often insufficient and the fixation may fail (see R. Wittenberg et al., “Importance of bone mineral density in instrumented spine fusions.” Spine, vol. 16, no. 6, pp. 647-652, 1991) (see K. Okuyama et al., “Stability of transpedicle screwing for the osteoporotic spine. an in vitro study of the mechanical stability.” Spine, vol. 18, no. 15, pp. 2240-2245, 1993) (see L. Weiser et al., “Insufficient stability of pedicle screws in osteoporotic vertebrae: biomechanical correlation of bone mineral density and pedicle screw fixation strength,” European Spine Journal, vol. 26, no. 11, pp. 2891-2897, 2017). As conceptually shown in FIG. 9N, this failure can mainly be attributed to the lack of dexterity in existing rigid drilling instruments, linear trajectory of the rigid pedicle screws, and the complex anatomy of vertebra forcing the surgeon to implant the screw within low BMD osteoporotic regions (see C. L. Goldstein et al., “Surgical management of spinal conditions in the elderly osteoporotic spine,” Neurosurgery, vol. 77, no. suppl 1, pp. S98-S107, 2015). With access to new areas of the vertebra, using novel flexible drilling instruments, surgeons would be able to optionally implant flexible pedicle screws into high BMD regions (shown in FIG. 9N), thus improving the quality of spinal fusion surgeries (see F. Alambeigi et al., “Inroads toward robot-assisted internal fixation of bone fractures using a bendable medical screw and the curved drilling technique,” in 2018 7th IEEE International Conference on Biomedical Robotics and Biomechatronics (Biorob). IEEE, 2018, pp. 595-600) (see F. Alambeigi et al., “On the use of a continuum manipulator and a bendable medical screw for minimally invasive interventions in orthopedic surgery,” IEEE transactions on medical robotics and bionics, vol. 1, no. 1, pp. 14-21, 2019). This demands a flexible yet structurally strong robot/instrument that is able to drill a pre-planned smooth curved path into a patient's hard tissue (bone) quickly, reliably, and without disrupting the surgeon's procedural workflow. Providing a simultaneous balance between the structural stiffness and dexterity of a flexible drilling robot while drilling a pre-planned smooth curved path is the essential challenge in designing these robotic systems. Of note, this design requirement is of considerable importance to avoid buckling and drilling failure when external interaction forces are exerted on the robotic system (see F. Alambeigi et al., “Toward robot-assisted hard osteolytic lesion treatment using a continuum manipulator,” in 2016 38th Annual International Conference of the IEEE Engineering in Medicine and Biology Society (EMBC). IEEE, 2016, pp. 5103-5106) (see Y. Wang et al., “Design and experimental validation of a miniaturized robotic tendon driven articulated surgical drill for enhancing distal dexterity in minimally invasive spine fusion,” IEEE/ASME Transactions on Mechatronics, 2021).

To overcome the aforementioned challenges, literature documents a few flexible drilling robots developed to improve clinicians' access within hard tissues. For instance, Alambeigi et al. utilized a tendon-driven continuum manipulator to develop a robotic system capable of drilling relatively smooth curved holes for treatment of femoral head osteonecrosis (see F. Alambeigi et al., “On the use of a continuum manipulator and a bendable medical screw for minimally invasive interventions in orthopedic surgery,” IEEE transactions on medical robotics and bionics, vol. 1, no. 1, pp. 14-21, 2019) (see F. Alambeigi et al., “A curved-drilling approach in core decompression of the femoral head osteonecrosis using a continuum manipulator,” IEEE Robotics and Automation Letters, vol. 2, no. 3, pp. 1480-1487, 2017). The system, however, suffers from several limitations including a lengthy drilling procedure, as long as 5-9 minutes, for cutting a small curved path with a maximum 35 mm length. This is mainly due to the lack of adequate structural stiffness and inherent compliance of the utilized continuum manipulator, increasing the risk of drilling failure and buckling at high insertion speeds (see F. Alambeigi et al., “Toward robot-assisted hard osteolytic lesion treatment using a continuum manipulator,” in 2016 38th Annual International Conference of the IEEE Engineering in Medicine and Biology Society (EMBC). IEEE, 2016, pp. 5103-5106). More importantly, due to the under-actuation of tendon-driven continuum manipulators, this system is unable to be steered along an arbitrary pre-planned drilling trajectory (see Y. Wang et al., “Design and experimental validation of a miniaturized robotic tendon driven articulated surgical drill for enhancing distal dexterity in minimally invasive spine fusion,” IEEE/ASME Transactions on Mechatronics, 2021). The performed experiments solely demonstrated the feasibility of drilling a few curved trajectories based on predetermined drilling parameters (i.e., insertion and rotational speeds and actuation tensions). In other words, the utilized tension-based control algorithm cannot provide an accurate active control algorithm to steer the robot along a desired pre-planned trajectory independent of the hard tissue properties. To partially address these limitations, recently, Ma et al. developed a handheld version of the mentioned robotic system (see J. H. Ma et al., “An active steering hand-held robotic system for minimally invasive orthopaedic surgery using a continuum manipulator,” IEEE Robotics and Automation Letters, vol. 6, no. 2, pp. 1622-1629, 2021). Nevertheless, similar to the previous study, stiffness and bending behavior of the drilling robot are limited to the stiffness of the utilized continuum manipulator and the maximum loading capacity of the actuation tendons. Moreover, due to the inherent compliance and underactuation of tendon-driven continuum manipulators, steering of this handheld drilling system (i.e., simultaneous control of insertion and bendingDegrees-of-Freedom (DoF)) is highly unintuitive and depends on the user's expertise limiting its application for accurate surgical procedures.

In an effort toward addressing the above-mentioned limitations, Wang et al. have recently proposed an actively controlled tendon-driven articulated surgical drilling system for spinal fixation interventions (see Y. Wang et al., “Design and experimental validation of a miniaturized robotic tendon driven articulated surgical drill for enhancing distal dexterity in minimally invasive spine fusion,” IEEE/ASME Transactions on Mechatronics, 2021) (see Y. Wang et al., “A handheld steerable surgical drill with a novel miniaturized articulated joint module for dexterous confined-space bone work,” IEEE Transactions on Biomedical Engineering, pp. 1-1, 2022). Although, compared to the conventional rigid drilling instruments, this drill grants more access to the areas within vertebra, it does not allow for a smooth curved trajectory through the bone for implantation of flexible pedicle implants. In other words, due to the use of a rigid shaft and an articulated wrist/hinge, the drilled trajectory is restricted to multi-segment straight/linear paths into the bone.

Screw implants are commonly used to stabilize bone fractures, reconstruct bone after tumor resection or destruction from infection, and treat congenital and acquired degenerative diseases. Screw fixation usually inserts rigid bone screws through strong cortical bone and into the more porous cancellous bone. The screws can then be rigidly connected with locking rods to ideally provide a stable fixation and load sharing feature before a robust bone fusion or healing occurs. However, screw fixation suffers from various types of complications and failures, including but not limited to screw misplacement, screw fracture, bone fracture, and loosening and pullout of screw implants. In particular, while loosening and pullout of screw implants is a prevalent problem in osteoporotic bone, it is also a common occurrence in bones with normal and healthy bone mineral density (BMD).

The reasons for the inadequacy of screw implants in bone are manifold. Screw implant sites in bone must deal with narrow and confined anatomical constraints, limiting the angles of approach for the screws. Nerves and blood vessels also must be avoided from the screw path. Additional obstacles are regions of low BMD. Fixation strength and quality of screw implant fixation directly depend on the BMD of an implant site. Traditional drilling instruments and screws are rigid and lack the sufficient dexterity to navigate the aforementioned anatomical constraints, limiting implant trajectories to linear paths that often lead to screw misplacement and nerve injury and necessarily cross low BMD regions.

Other devices have attempted to solve the abovementioned problems but are limited in their applications and solutions. One such device is described in U.S. Pat. No. 11,007,010, which includes a J-stylet configured for malleting with a D-shaped cross sectional springboard end manufactured via mechanical grinding, but this device is unable to drill curved trajectories, drill branched trajectories, or perform cavity cutting due to its lack of a flexible drilling apparatus. Other such devices are described in U.S. Pat. Nos. 9,517,077, 9,808,294, and 10,368,921, which include ties placed through facet joints, but these devices are only for use in facet joint locations of the spine.

Thus, there is a need in the art for improved devices and methods for implant fixation in bone that are adapted for a subject's bone mineral density. Particularly, there is a need for devices that can drill in long curved trajectories, branched trajectories, and/or cavity cutting inside bones with complex curved anatomies such as the pelvis and vertebrae, for example. The present invention meets this need.

### SUMMARY OF THE INVENTION

Some embodiments of the invention disclosed herein are set forth below, and any combination of these embodiments (or portions thereof) may be made to define another embodiment.

In one aspect a drilling robot device comprises an outer tube including a proximal end, a distal end, and a first concentric through hole centered on a longitudinal axis extending from the proximal end to the distal end, at least one inner tube movably nested within the outer tube including a proximal end, a distal end, and a second concentric through hole centered on the longitudinal axis, wherein the at least one nested inner tube is concentric with the outer tube, and a flexible drive shaft including a proximal end, a distal end and a tool tip positioned at the distal end, wherein the flexible drive shaft extends through the second concentric through hole of the at least one inner tube, and is configured to provide a rotational torque to the tool tip.

In one embodiment, the flexible drive shaft comprises a torque coil. In one embodiment, the at least one inner tube is curved. In one embodiment, the at least one inner tube comprises a curved portion and a linear portion. In one embodiment, the at least one inner tube is pre-treated to follow a preset curvature. In one embodiment, the radius of the preset curvature is 5 to 200 mm. In one embodiment, the at least one inner tube is heat-treated. In one embodiment, the at least one inner tube is differentially heat-treated. In one embodiment, the at least one inner tube comprises nitinol.

In one embodiment, the outer tube has a diameter of 1 to 20 mm, a wall thickness of 0.05 to 4 mm, and a length of 5 to 500 mm and the first concentric through hole has a diameter of 1 to 20 mm. In one embodiment, the at least one inner tube has a diameter of 1 to 20 mm, a wall thickness of 0.05 to 5 mm, and a length of 5 to 500 mm and the second concentric through hole has a diameter of 1 to 20 mm.

In another aspect, a drilling robot system comprises a manipulation system configured to provide a manipulative force and a drilling torque, and a drilling robot device movably connected to the manipulation system and configured to receive the manipulative force comprising an outer tube including a proximal end, a distal end, and a first concentric through hole centered on a longitudinal axis extending from the proximal end to the distal end, at least one inner tube movably nested within the outer tube including a proximal end, a distal end, and a second concentric through hole centered on the longitudinal axis, wherein the at least one nested inner tube is concentric with the outer tube, and a flexible drive shaft including a proximal end, a distal end and a tool tip positioned at the distal end, wherein the flexible drive shaft extends through the second concentric through hole of the at least one inner tube, and is configured to provide a rotational torque to the tool tip.

In one embodiment, the manipulation system comprises a handheld manipulator. In one embodiment, the manipulation system comprises a robotic arm. In one embodiment, the robotic arm is configured to perform a robotic assisted procedure. In one embodiment, the robotic assisted procedure comprises a surgical procedure.

In one embodiment, the manipulation system comprises a drill motor configured to provide a torque to the tool tip via the flexible drive shaft, and a hand operated linear slide to translationally actuate the at least one inner tube, flexible drive shaft and tool tip.

In one embodiment, the manipulation system comprises a drill motor configured to provide a torque to the tool tip via the flexible drive shaft, and a translational actuation motor configured to actuate a translational actuation mechanism to translationally actuate the inner tube, flexible drive shaft and tool tip.

In one embodiment, the manipulation system comprises a drill spline shaft configured to transfer torque provided by a drill motor to the tool tip via a drill carriage and the flexible drive shaft, a rotational actuation motor configured to rotate the inner tube via a rotational actuation spline shaft and a main housing unit, and a translational actuation motor configured to linearly actuate the drilling robot device via rotating a translational actuation lead screw and the main housing unit.

In another aspect, a drilling method comprises providing a manipulation system configured to provide a manipulative force and a drilling torque, providing a concentric tube drilling robot device movably connected to the manipulation system and configured to receive the manipulative force, and drilling a trajectory via a combination of the manipulative force, drilling torque, and a curvature inherent to the concentric tube drilling robot device.

In one embodiment, the concentric tube drilling robot device comprises an outer tube including a proximal end, a distal end, and a first concentric through hole centered on a longitudinal axis extending from the proximal end to the distal end, at least one inner tube movably nested within the outer tube including a proximal end, a distal end, and a second concentric through hole centered on the longitudinal axis, wherein the at least one nested inner tube is concentric with the outer tube, and a flexible drive shaft including a proximal end, a distal end and a tool tip positioned at the distal end, wherein the flexible drive shaft extends through the second concentric through hole of the at least one inner tube, and is configured to provide a rotational torque to the tool tip.

In one embodiment, the manipulation system comprises at least one of a handheld manipulator and a robotic arm. In one embodiment, the method further comprises characterizing a target bone tissue including identifying regions of osteoporotic bone and bone with low mineral density, and forming the drilling trajectory based on the characterization. In one embodiment, the drilling trajectory is configured to avoid the identified regions of osteoporotic bone and bone with low mineral density.

In one embodiment, the drilling trajectory is configured to follow a three dimensional curved, long, and complex anatomy in which nerves and vessels need to be avoided during the drilling procedure. In one embodiment, the step of characterizing the target bone tissue comprises the steps of performing one or more quantitative computed tomography (QCT) scans on the target bone tissue, converting the one or more QCT scans into a three-dimensional finite element model of the target bone tissue, and demarcating osteoporotic regions or low bone mineral density regions in the three-dimensional finite element model.

In one embodiment, the drilled trajectory comprises at least one of a J-shaped trajectory, an S-shaped trajectory, a U-shaped trajectory, a combination of a linear and a curved trajectory, a multi-segment trajectory, a multiple J-shaped branch trajectory, and a minimally invasive cavity cutting trajectory.

In one embodiment, the drill crosses between adjacent vertebrae to connect two drilling access points to allow for a flexible fixation device to pass traverse through them.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary drilling robot device in accordance with some embodiments.

FIG. 2 depicts a first view of an exemplary drilling robot system in accordance with some embodiments.

FIG. 3 depicts a second view of the exemplary drilling robot system in accordance with some embodiments.

FIG. 4 depicts a first view of another exemplary drilling robot system in accordance with some embodiments.

FIG. 5 depicts a second view of the exemplary drilling robot system in accordance with some embodiments.

FIGS. 6A-6C depict another exemplary drilling robot system in accordance with some embodiments.

FIG. 7 is a flowchart depicting an exemplary drilling method in accordance with some embodiments.

FIG. 8 depicts an exemplary drilling trajectory in accordance with some embodiments.

FIGS. 9A-9R depict exemplary applications of a drilling robot device in accordance with some embodiments.

FIGS. 10A-10D depict an exemplary experimental setup of a drilling robot system in accordance with some embodiments.

FIG. 11 depicts an exemplary experimental result of a drilling robot system in accordance with some embodiments.

FIG. 12 depicts an exemplary experimental result of a drilling robot system in accordance with some embodiments.

FIG. 13 depicts an exemplary experimental result of a drilling robot system in accordance with some embodiments.

FIG. 14 depicts examples of experimentally heat-treated inner tubes of different curvatures over curved trajectories in a 3D printed L3 vertebra in accordance with some embodiments.

FIG. 15 shows tables of experimental results in accordance with some embodiments.

FIG. 16 shows an experimental setup used to evaluate performance of the system in accordance with some embodiments.

FIG. 17 show plaster (right) and laser scanned (left) representations and measurements of the experimental drilled out-of-plane Branch J-shape drilling tunnels in accordance with some embodiments.

FIG. 18 shows experimental X-ray images from animal drilling experiments performed with the 71.1 mm NiTi tube in accordance with some embodiments.

FIG. 19 shows experimental average magnitude of the drilling force throughout the drilling procedure on both Sawbone and animal bone samples captured by the force/torque load cell in accordance with some embodiments.

FIG. 20 shows experimental components of the measured and smoothed forces during animal bone drilling performed with the 71.1 mm radius of curvature in accordance with some embodiments.

FIG. 21 shows an experimental setup used to evaluate performance of the system in accordance with some embodiments.

FIG. 22 shows experimental NiTi steering guides and the flexible shaft of the exemplary system in accordance with some embodiments.

FIG. 23 shows an experimental X-ray view of a U-shape trajectory test performed with a 39.9 mm steering guide in PCF 10 Sawbone in accordance with some embodiments.

FIG. 24 shows a theoretical representation of the ideal cavity volume removed by the system in accordance with some embodiments.

FIG. 25 shows experimental X-ray images showing progression of a test by moving the system through free space with a 39.9 mm radius steering guide in accordance with some embodiments.

FIG. 26 shows experimental 3D renderings (left) of actual cavity drilling models (right) in accordance with some embodiments.

FIG. 27 shows experimental components of both the measured and smooth forces during a pure rotational cavity drilling test performed in 10 PCF Sawbone with the 71.1 mm steering guide in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of concentric tube drilling. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a concentric tube drilling robot device, system and method.

The present invention relates to a concentric tube drilling robot device and system with associated drilling method, which can be utilized for a variety of applications such as surgical interventions requiring complex 3D curved drilling trajectories in hard tissues, for example. The device and system provide better drilling trajectory customization, while allowing for smaller surgical incisions, less trauma and increased surgical implant performance by being able to avoid obstacles and target desired tissues.

One goal of the disclosed devices, systems methods is to enhance fusion stability in an osteoporotic vertebra and avoid a spinal fixation failure. The design is inspired by concentric tube robots (see P. E. Dupont et al., “Design and control of concentric-tube robots,” IEEE Transactions on Robotics, vol. 26, no. 2, pp. 209-225, 2009) (see R. J. Webster III and B. A. Jones, “Design and kinematic modeling of constant curvature continuum robots: A review,” The International Journal of Robotics Research, vol. 29, no. 13, pp. 1661-1683, 2010) (see J. Burgner-Kahrs et al., “Continuum robots for medical applications: A survey,” IEEE Transactions on Robotics, vol. 31, no. 6, pp. 1261-1280, 2015). The disclosed system can provide quick, reliable, and accurate access to high BMD regions within the vertebral body by creating one or multiple completely-smooth curved drilling tunnels. In contrast to conventional applications of concentric tube robots demanding a free space to navigate through constrained anatomies with deformable tissues and avoid interaction with their environment, the disclosed system balances the innate compliance and ability to navigate in tight spaces of concentric tube robots with the adequate structural strength required to directly interact with bone tissue without experiencing unwanted deformation or buckling (see J. Burgner-Kahrs et al., “Continuum robots for medical applications: A survey,” IEEE Transactions on Robotics, vol. 31, no. 6, pp. 1261-1280, 2015) (see P. E. Dupont et al., “A decade retrospective of medical robotics research from 2010 to 2020,” Science Robotics, vol. 6, no. 60, p. eabi8017, 2021) (see P. E. Dupont et al., “Continuum robots for medical interventions,” Proceedings of the IEEE, vol. 110, no. 7, pp. 847-870, 2022).

Moreover, in some embodiments, thanks to the utilized preshaped nitinol (NiTi) tubes, the robotic system decouples the control of bending and insertion DoFs to provide a more intuitive and easy-to-steer procedure along the desired drilling trajectory. In other words, the bending direction has been pre-programmed to the system and solely the insertion DoF needs to be actively controlled during the procedure. This outstanding feature completely addresses the mentioned challenge in active steering of the previous robotic systems.

In some embodiments, the robotic system is capable of controlling both the insertion and rotation DoFs simultaneously to address the mentioned challenge in active steering of the previous robotic systems.

The performance and repeatability of the disclosed systems have been quantitatively assessed under different experimental conditions while drilling different fully smooth J-shape trajectories within simulated bone materials and animal bone samples.

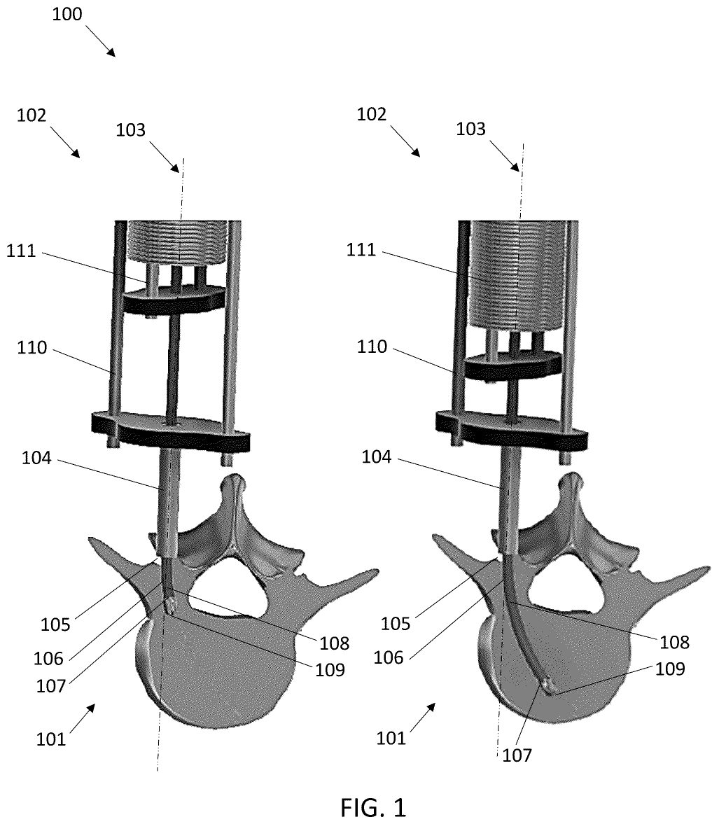

FIG. 1 shows an exemplary drilling robot device 100 in accordance with some embodiments. In some embodiments, the device 100 comprises an outer tube 104 including a proximal end 102, a distal end 101, and a first concentric through hole 105 centered on a longitudinal axis 103 extending from the proximal end 102 to the distal end 101. In some embodiments, the device 100 further comprises at least one inner tube 106 movably nested within the outer tube 104 including a proximal end 102, a distal end 101, and a second concentric through hole 107 centered on the longitudinal axis 103, wherein the at least one nested inner tube 106 is concentric with the outer tube 104. In some embodiments, the device 100 further comprises a flexible drive shaft 108 including a proximal end 102, a distal end 101 and a tool tip 109 positioned at the distal end 101, wherein the flexible drive shaft 108 extends through the second concentric through hole 107 of the at least one inner tube 106, and is configured to provide a rotational torque to the tool tip 109. In some embodiments, the device 100 further comprises a housing 110 connected to the outer tube 104, and at least one actuation mechanism 111 connected to the inner tube 106, and configured to linearly move the at least one inner tube 106 in proximal and distal directions relative to the outer tube 104. In some embodiments, a plurality of actuation mechanisms 111 are connected to the at least one inner tube 106 and the at least one outer tube 104, and are configured to linearly and/or rotatably move the at least one inner tube 106 and the at least one outer tube 104.

In some embodiments, the flexible drive shaft 108 comprise a torque coil or other suitable flexible transmission coil. In some embodiments, the at least one inner tube 106 is curved. In some embodiments, the at least one inner tube 106 comprises a curved portion and a linear portion. In some embodiments, the at least one inner tube 106 is pre-treated to follow a preset curvature, wherein the radius of the preset curvature is 5 to 200 mm. In some embodiments, the at least one inner tube is heat-treated or differentially heat-treated.

In some embodiments, the preset curvature decouples the control of bending and insertion degrees of freedom to provide a more intuitive and easy-to-steer procedure along the desired drilling trajectory. Furthermore, the bending direction has been pre-programmed to the device 100 and only the insertion degree of freedom needs to be actively controlled during the procedure.

In some embodiments, the outer tube 104 comprises at least one of nitinol, stainless steel, titanium, a biocompatible material, or other suitable material or combination thereof. In some embodiments, the outer tube 104 has a diameter of 1 to 20 mm, a wall thickness of 0.05 to 4 mm, and a length of 5 to 500 mm. In some embodiments, the first concentric through hole has a diameter of 1 to 20 mm.

In some embodiments, the at least one inner tube 106 comprises at least one of nitinol, stainless steel, titanium, and a biocompatible material. In some embodiments, the at least one inner tube 106 has a diameter of 1 to 20 mm, a wall thickness of 0.05 to 5 mm, and a length of 5 to 500 mm. In some embodiments, the second concentric through hole has a diameter of 1 to 20 mm.

In some embodiments, each of the at least one inner tube 106 can comprise the same materials or different materials, as well as the same dimensions or different dimensions. Inner tubes 106 that are nested and telescoping have outer and inner diameters that are appropriately sized to fit within each other, as would be understood by persons having ordinary skill in the art. In some embodiments, the nested inner tubes 106 are tapered.

In some embodiments, when the tubes (104, 106) are inserted into one another their curvatures are combined and allow for the tool tip 109 of the device 100 to be manipulated in space with a rotation and translation of the tubes (104, 106).

In some embodiments, the outer tube 104 is static while the at least one inner tube 106 is configured as a steering cannula able to house flexible cutting tools. When the device is in a first operating configuration the inner tube 106 is fully within the out tube 104, and the inner tube 106 is constrained to the straight geometry of the outer steel tube 104. As the inner tube 106 is pushed forward out of the out tube 104 to a second operation configuration, the inner tube 106 returns to its pre-programmed heat-treated shape. The tool tip 109 is also simultaneously guided by the inner tube 106 to follow the path dictated by the preset shape along the planned drilling trajectory and to not deviate from it.

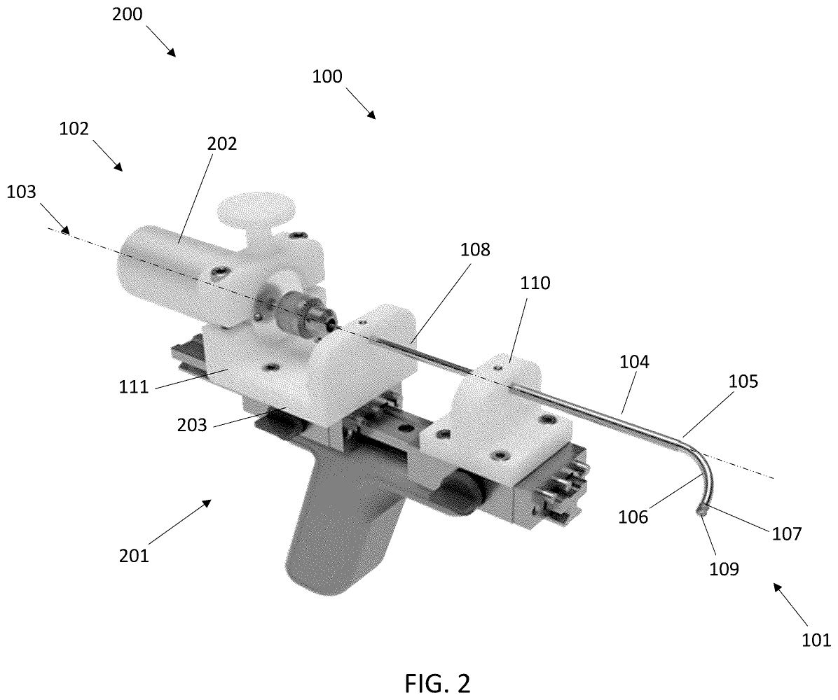

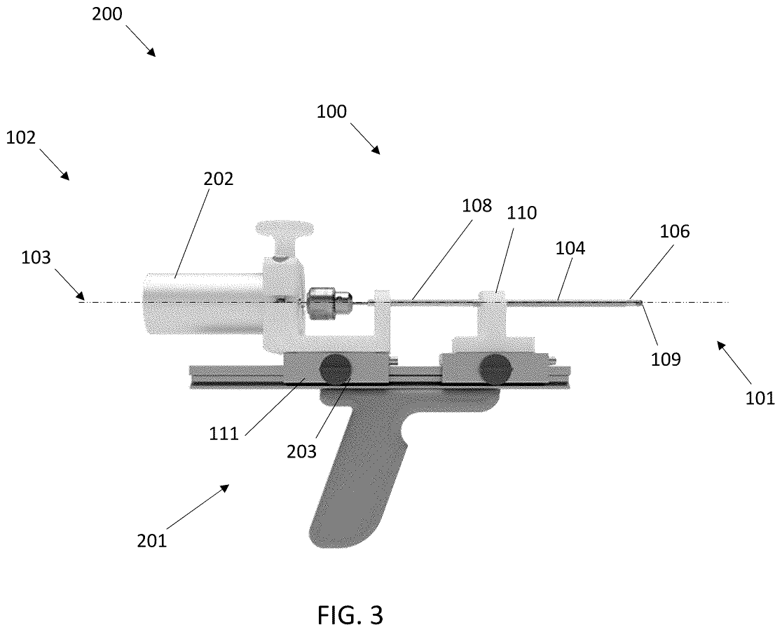

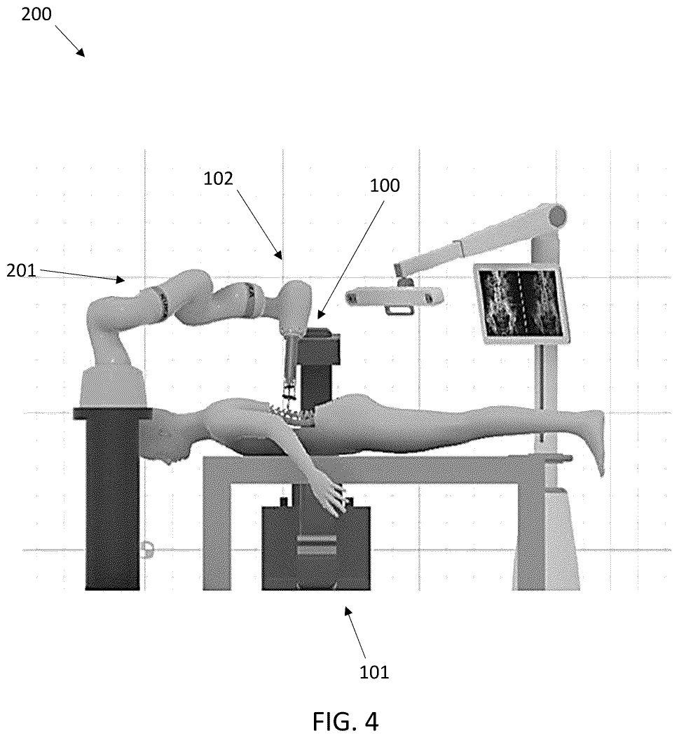

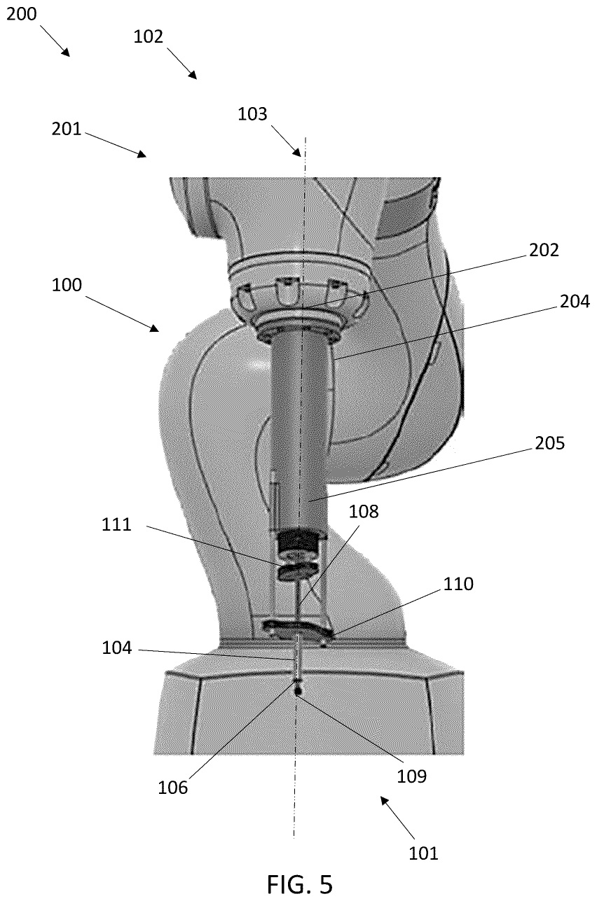

FIGS. 2 and 3 show views of an exemplary drilling robot system 200 in accordance with some embodiments. FIGS. 4 and 5 show views of another exemplary drilling robot system 200 in accordance with some embodiments.

The system 200 comprises a manipulation system 201 configured to provide a manipulative force and a drilling torque and a drilling robot device 100 as described above, movably connected to the manipulation system 200 and configured to receive the manipulative force. In some embodiments, the manipulative force can be a linear force, a rotational force, and/or a combination thereof. In the embodiment shown in FIGS. 2-3, the manipulation system 201 comprises a handheld manipulator. In the embodiment shown in FIGS. 4-5, the manipulation system 201 comprises a robotic arm. In some embodiments, the robotic arm is configured to perform a robotic assisted procedure, such as a surgical procedure. In some embodiments, the surgical procedure can include, for example, orthopedic and neurosurgical interventions requiring curved drilling of hard tissues. In some embodiments, the manipulative force and drilling torque are provided by one or more motors.

In some embodiments, the outer tube 104 and the at least one inner tube 106 are each independently controlled, thus allowing for the tool tip 109 to be manipulated into different regions of 3D space. In some embodiments, S-shaped trajectories and/or J-shaped trajectories can be drilled with the system 200 via a combination of pre-curved inner tubes 106. In some embodiments, cavities can be cut inside of a tissue via a simultaneous rotation of the inner tube 106 and drilling done by the tool tip 109. In some embodiments, the drilling is performed as a minimally invasive procedure. In some embodiments, the tool tip 109 is swappable. The tool tip 109 can comprise at least one of a threaded tap to prepare for curved screw insertion, a fenestrated grasper, an additional irrigation source or drain for inside the hole, an endoscope, a drill bit, and any other suitable tool or combination thereof.

Referring to FIGS. 2-3, in some embodiments, the manipulation system 201 includes a drill motor 202 configured to provide a torque to the tool tip 109 via the flexible drive shaft 108, and a hand operated linear slide 203 to actuate the insertion of the inner tube 106, flexible drive shaft 108 and tool tip 109 of the drilling device 100.

Referring to FIGS. 4-5, in some embodiments, the manipulation system 201 includes a drill motor 202 configured to provide a torque to the tool tip 109 via the flexible drive shaft 108, and a translational actuation motor 204 configured to actuate a translational actuation mechanism 205, such as a lead screw, to actuate the insertion of the inner tube 106, flexible drive shaft 108 and tool tip 109 of the drilling device 100.

In other embodiments, the system 200 can be scaled up in size for use in industries including manufacturing, metalworking, woodworking, mining, construction and other suitable industries.

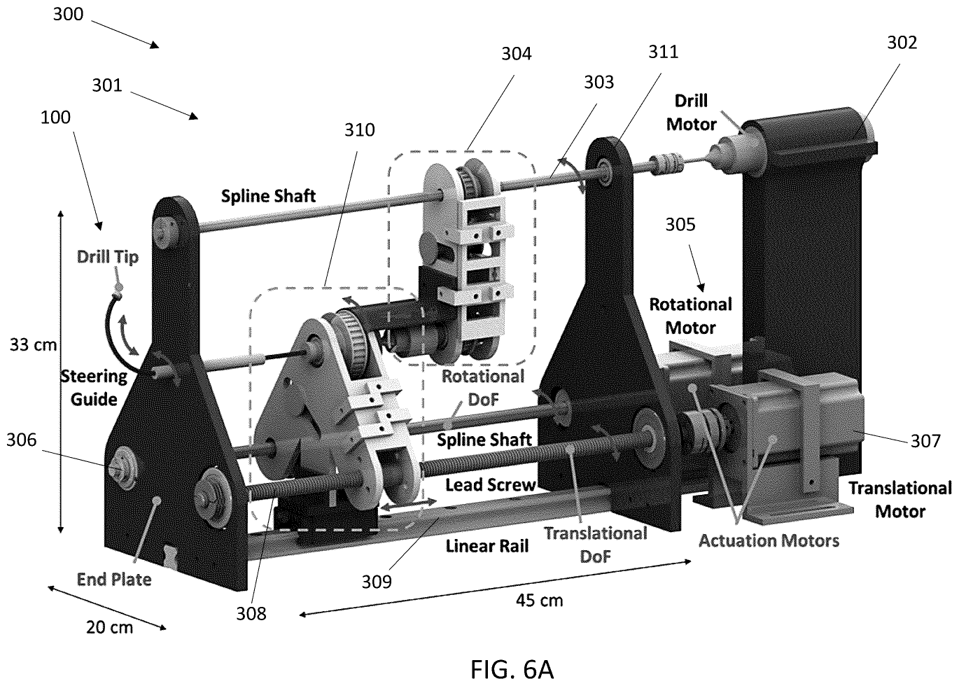

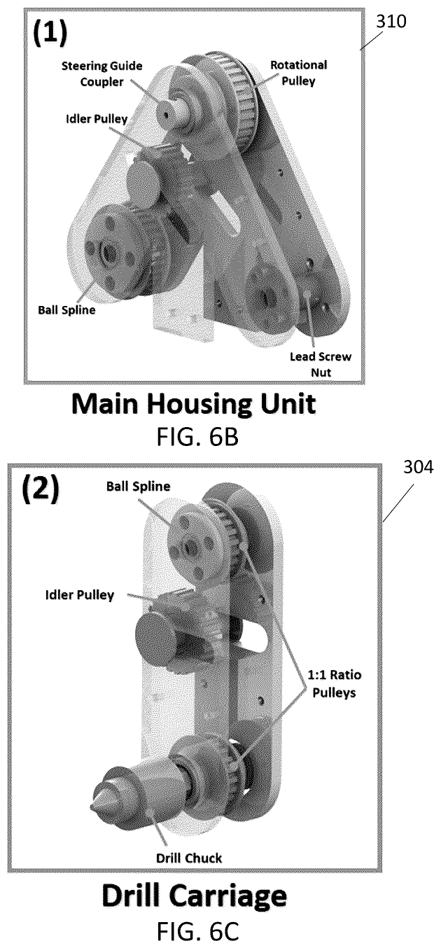

FIGS. 6A-6C shows another exemplary drilling robot system 300 in accordance with some embodiments. The system 300 comprises a manipulation system 301 configured to provide manipulative forces and a drilling torque, and a drilling robot device 100 as described above, movably connected to the manipulation system 300 and configured to receive the manipulative forces. In some embodiments, the manipulative forces can be a linear force, a rotational force, and/or a combination thereof. In the embodiment shown in FIGS. 6A-6C, the manipulation system 300 comprises a tri-motor system suitable for a tabletop implementation, for example. In some embodiments, the manipulative forces and drilling torque are provided by three motors.

In some embodiments, the outer tube 104 and the at least one inner tube 106 are each independently controlled, thus allowing for the tool tip 109 to be manipulated into different regions of 3D space. In some embodiments, S-shaped trajectories and/or J-shaped trajectories can be drilled with the system 300 via a combination of pre-curved inner tubes 106. In some embodiments, cavities can be cut inside of a tissue via a simultaneous rotation of the inner tube 106 and drilling done by the tool tip 109. In some embodiments, the drilling is performed as a minimally invasive procedure. In some embodiments, the tool tip 109 is swappable. The tool tip 109 can comprise at least one of a threaded tap to prepare for curved screw insertion, a fenestrated grasper, an additional irrigation source or drain for inside the hole, an endoscope, a drill bit, and any other suitable tool or combination thereof.

In some embodiments, the system 300 comprises an inverted Y-shaped configuration. In some embodiments, the system 300 includes a drill spline shaft 303 configured to transfer torque provided by a drill motor 303 to the tool tip 109 via a drill carriage 304 and flexible drive shaft 108. In some embodiments, the system 300 includes a rotational actuation motor 305 configured to rotate the inner tube 106 via a rotational actuation spline shaft 306 and a main housing unit 310. In some embodiments, the system 300 includes a translational actuation motor 307 configured to linearly actuate the drilling device 100 via rotating a translational actuation lead screw 308 and the main housing unit 310. In some embodiments the main housing unit 310 is supported on a linear rail 309 on which it slides. Support plates 311 can be used to support the rotational actuation spline shaft 306, the drill spline shaft 303, and the translational actuation lead screw 308. In some embodiments, a combination of torque inputs provided by the rotational actuation motor 305, the translational actuation motor 307, and/or the drill motor 302 are used to manipulate the drilling device 100 for drilling operations, insertion operations, and/or steering operations.

In other embodiments, the system 300 can be scaled up in size for use in industries including manufacturing, metalworking, woodworking, mining, construction and other suitable industries.

In some embodiments, system 200 or system 300 further comprises a computing system including a computing device configured to run software, and a user interface to interact with the computing device. In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

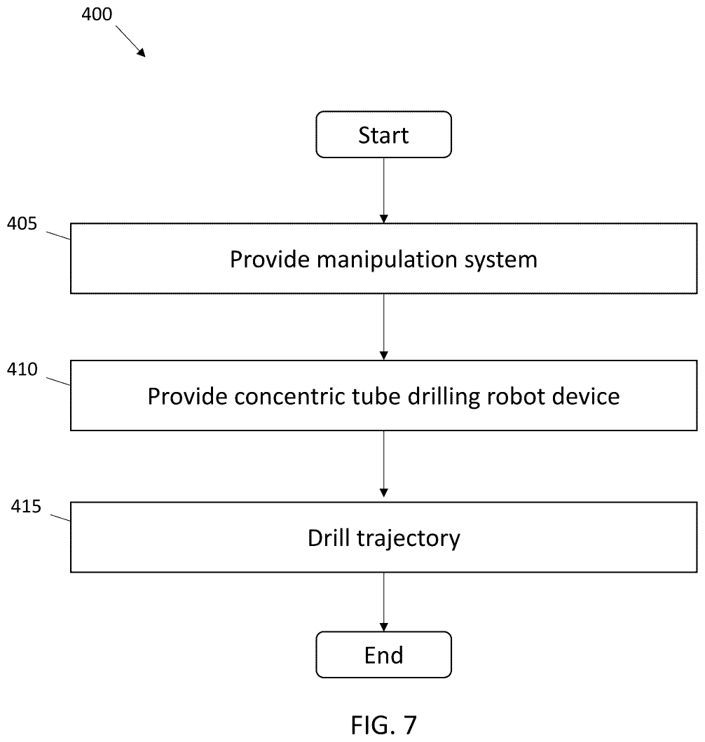

In one aspect, the present invention provides a method of drilling a preset drilling trajectory. FIG. 7 is a flowchart showing an example drilling method 400. The method 400 starts at Operation 405, where a manipulation system (201 or 301) is provided. In some embodiments, the manipulation system 201 comprises a handheld manipulator. In some embodiments, the manipulation system 201 comprises a robotic arm. In some embodiments, the manipulation system comprises a tabletop unit.

At Operation 410, a concentric tube drilling robot device 100 is provided. In some embodiments, the device 100 comprises an outer tube 104 including a proximal end 102, a distal end 101, and a first concentric through hole 105 centered on a longitudinal axis 103 extending from the proximal end 102 to the distal end 101. In some embodiments, the device 100 further comprises at least one inner tube 106 movably nested within the outer tube 104 including a proximal end 102, a distal end 101, and a second concentric through hole 107 centered on the longitudinal axis 103, wherein the at least one nested inner tube 106 is concentric with the outer tube 104. In some embodiments, the device 100 further comprises a flexible drive shaft 108 including a proximal end 102, a distal end 101 and a tool tip 109 positioned at the distal end 101, wherein the flexible drive shaft 108 extends through the second concentric through hole 107 of the at least one inner tube 106, and is configured to provide a rotational torque to the tool tip 109.

In one embodiment, the method 400 ends at Operation 415, where the drilling trajectory is drilled. In some embodiments, this drilling trajectory is preset. In some embodiments, the drilling trajectory is set by pretreating the at least one inner tube 106 to follow a preset curvature. In some embodiments, the pretreatment of the at least one inner tube 106 comprises a heat treatment and/or a differential heat treatment. In some embodiments, the drilling is performed by the manipulation system (201 or 301) supplying a manipulative force and a drilling torque to the drilling robot device 100. In some embodiments, the manipulative force can be a linear force, a rotational force, and/or a combination thereof.

In some embodiments, the system (200 or 300) can be controlled autonomously. In some embodiments, the system (200 or 300) can be controlled using a human-robotic interaction interface to tele-operate the drilling system using a device such as joystick, haptic device (e.g., Phantom Omni), a spatial 3D mouse, and/or a custom designed control interface that provides enough degrees of freedom to control the abovementioned degrees of freedom of the system (200 or 300).

In some exemplary embodiments, the method 400 is optionally preceded by characterizing a target bone tissue of a subject and forming an implantation drilling trajectory based on the characterization. In some embodiments, bone mineral density in a target tissue is measured using quantitative computed tomography (QCT) along with a calibration phantom positioned near a subject. The calibration phantom comprises regions of known Hounsfield units that appear darker with lower densities and lighter with higher densities. Using the calibration phantom, the bone mineral density of the target tissue can be quantified. QCT images are segmented, and a three-dimensional finite element model is constructed based on them such that each element of the model has the material property of the corresponding voxel in the QCT images. In some embodiments, the three-dimensional model is used to design and analyze a custom implantation drilling trajectory. Furthermore, it can demarcate osteoporotic regions and low bone mineral density regions of the target tissue for which possible implantation drilling trajectories may avoid. While osteoporotic regions may be defined as having a bone mineral density of less than 80 mg/cm³, it should be understood that any threshold may be used. For example, regions of bone mineral density may be characterized as low relative to the surrounding tissue, such that an optimal implantation drilling trajectory favors the higher density tissue over the lower density tissue, even if the lower density tissue has a bone mineral density greater than 80 mg/cm³. An optimal implantation drilling trajectory may avoid osteoporotic regions and low bone mineral density regions, resulting in minimized strain and improved implant pullout strength when compared to conventional linear screw implantation drilling paths that are unable to evade osteoporotic regions and low bone mineral density regions. In some embodiments, the pre-treatment to cause curvature of the at least one inner tube 106 is configured based on the characterization of the bone tissue.

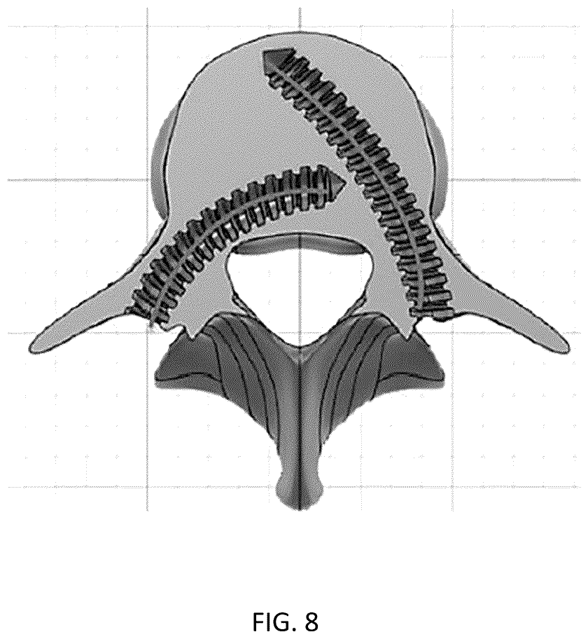

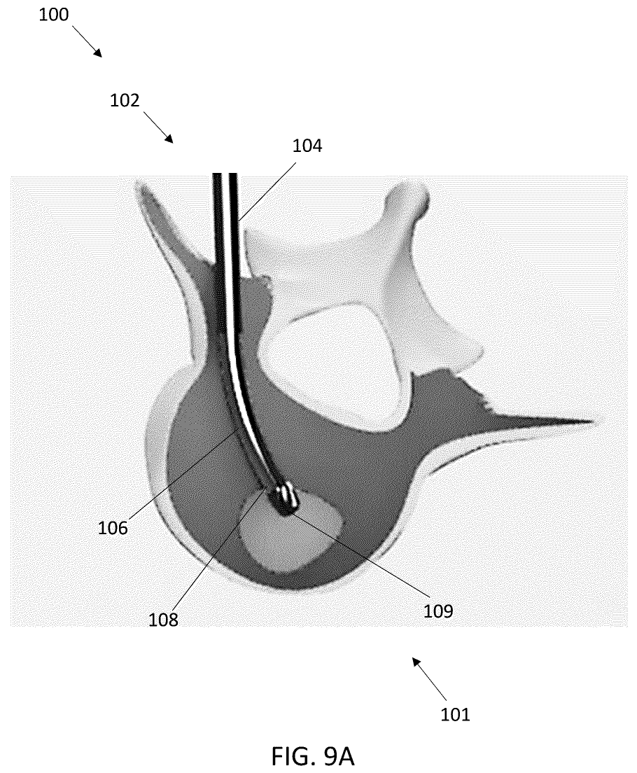

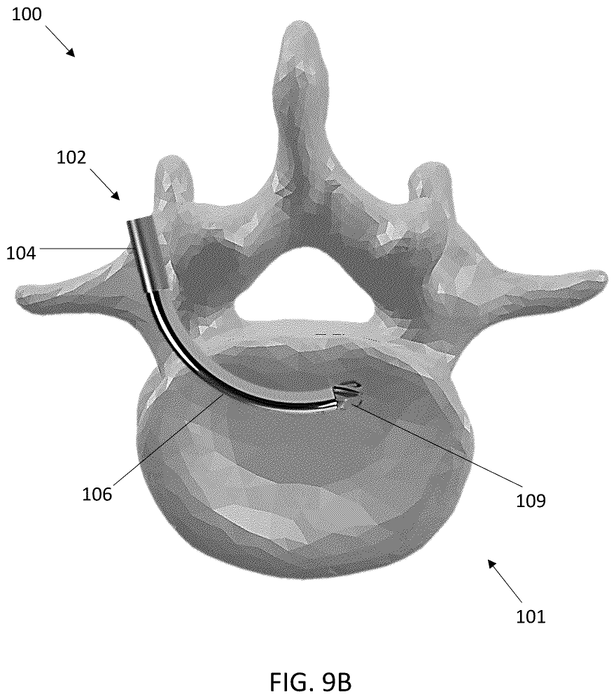

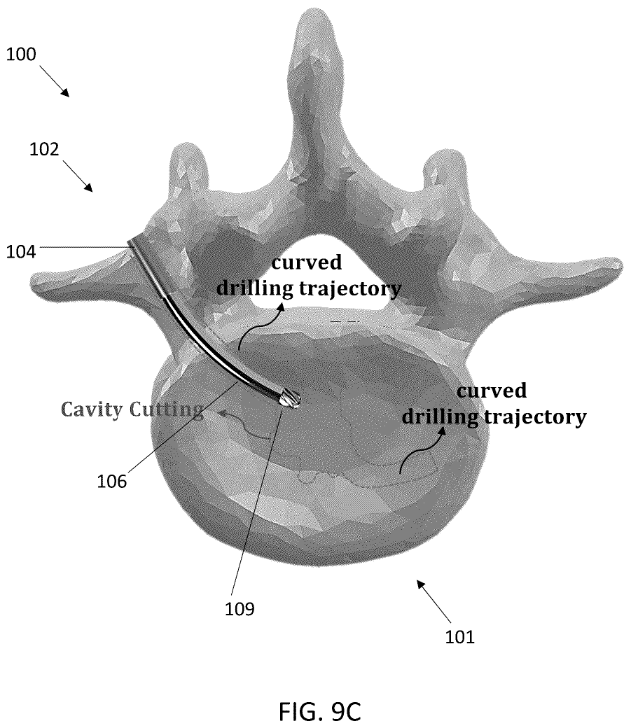

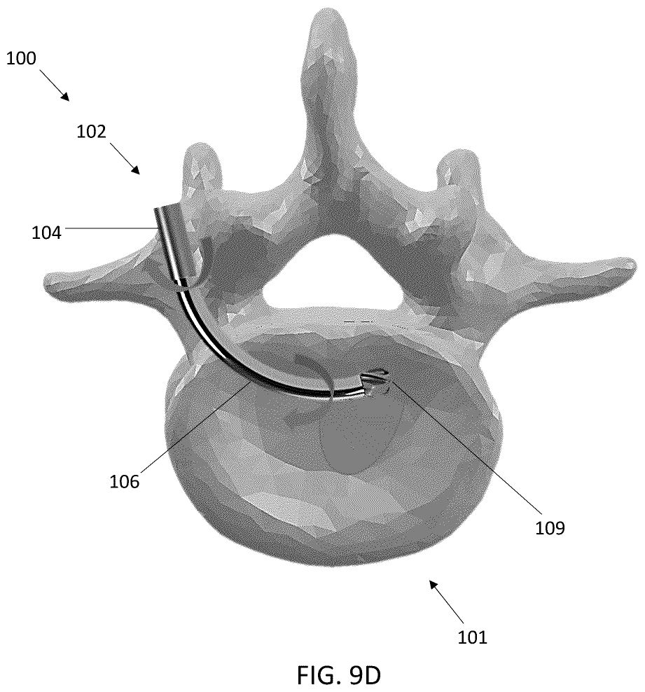

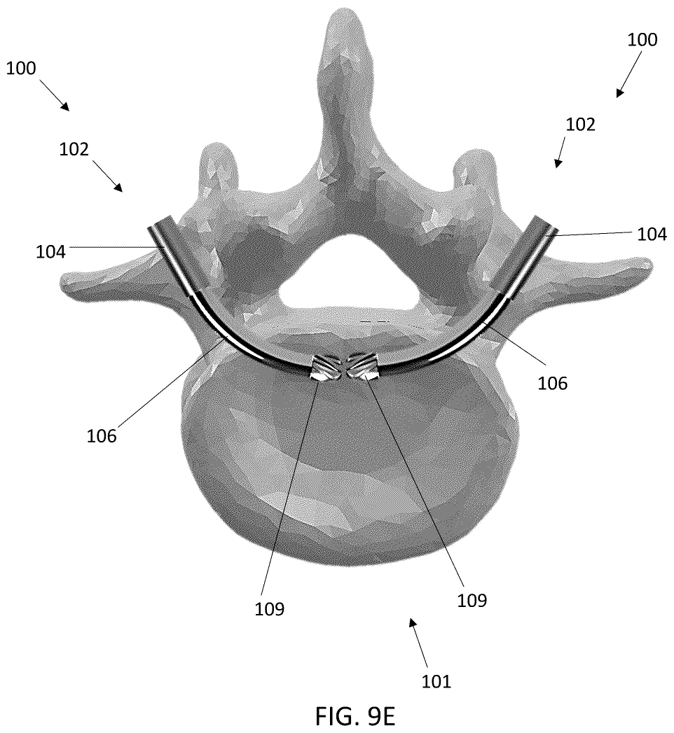

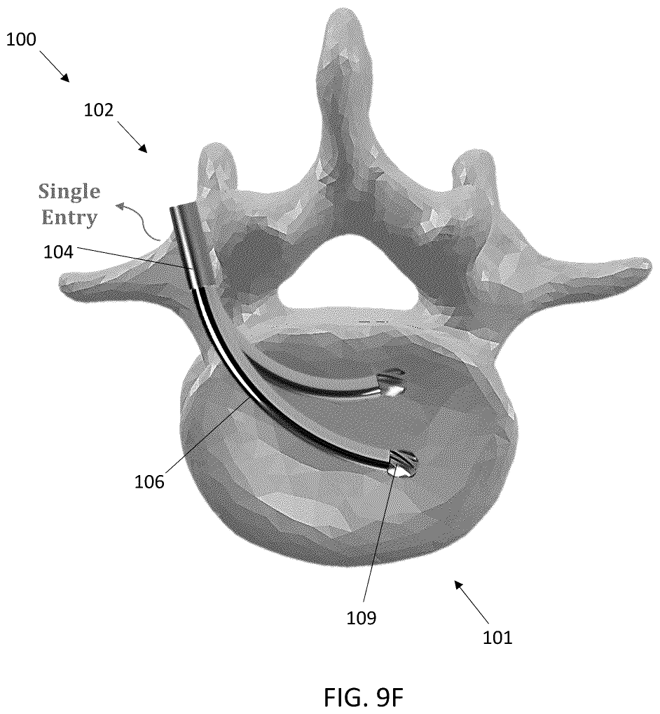

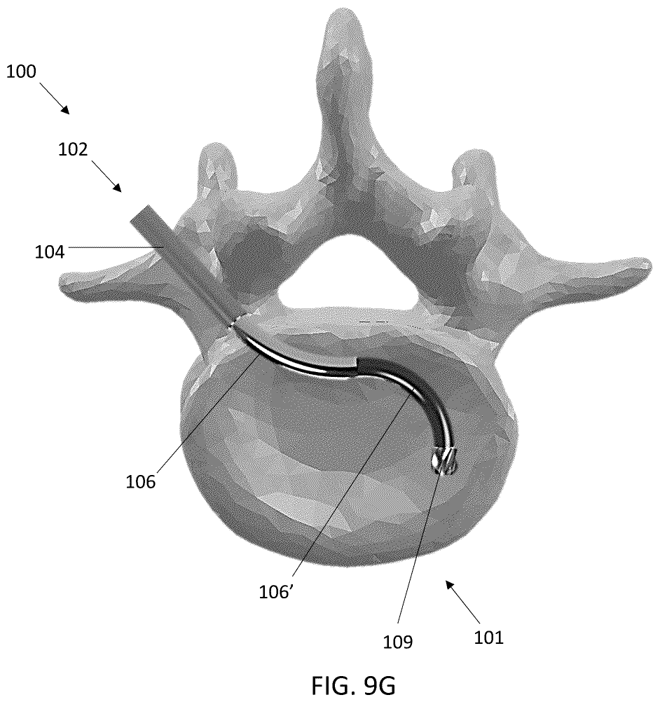

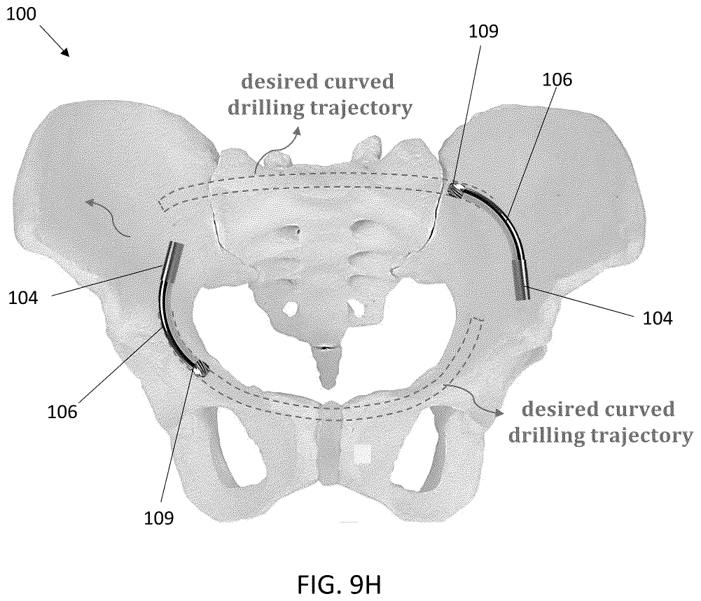

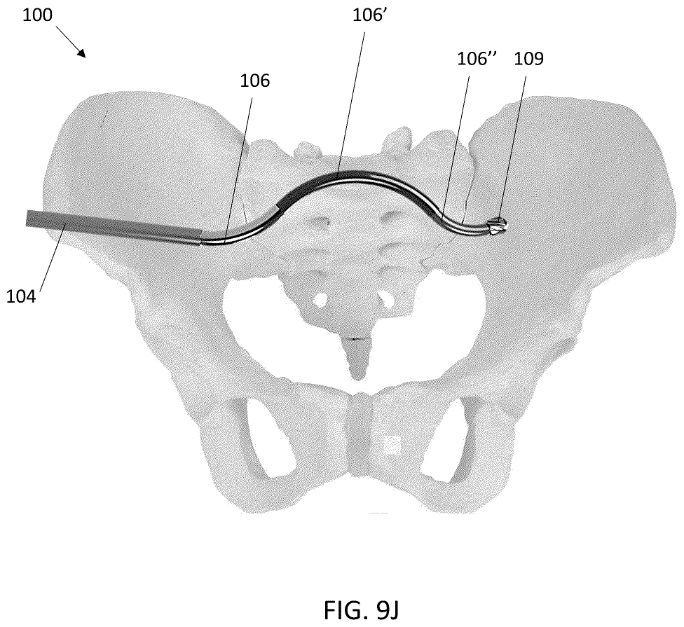

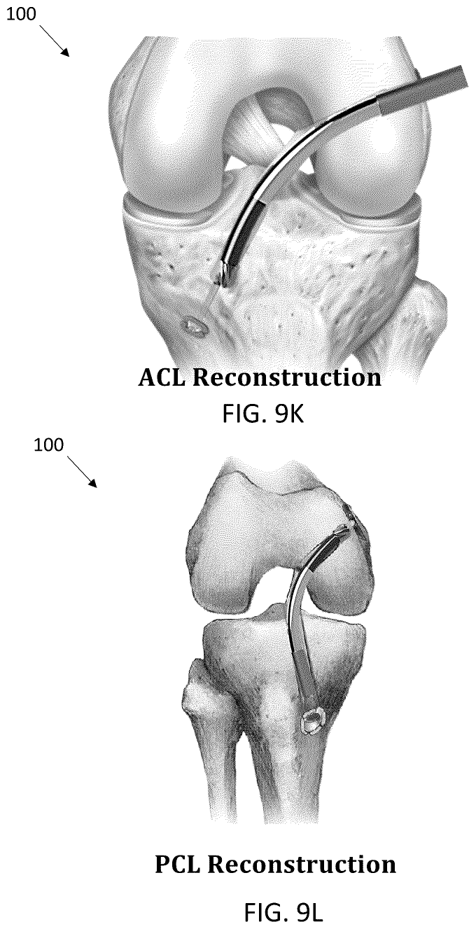

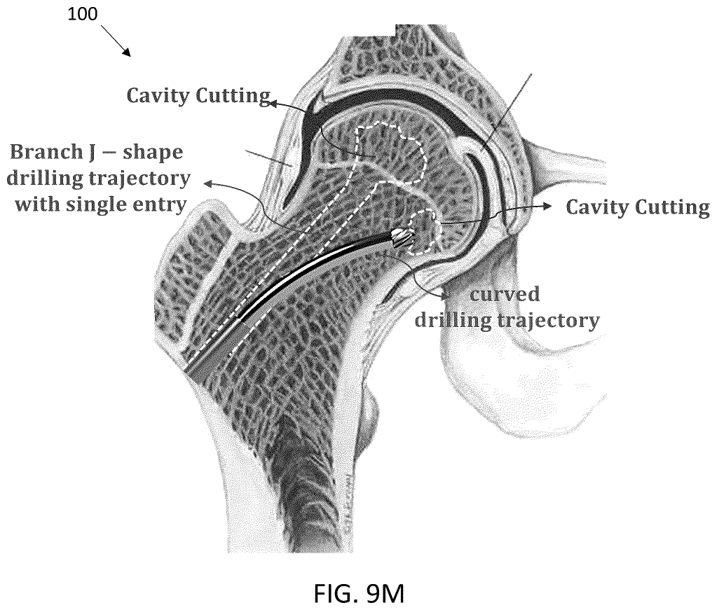

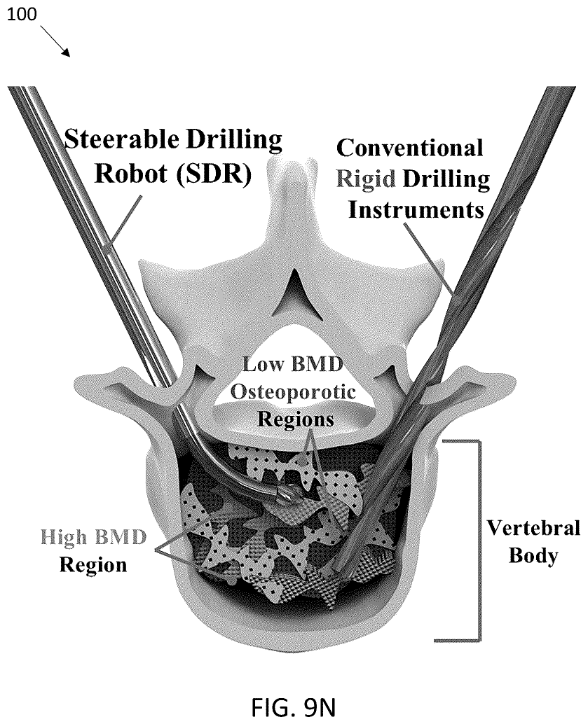

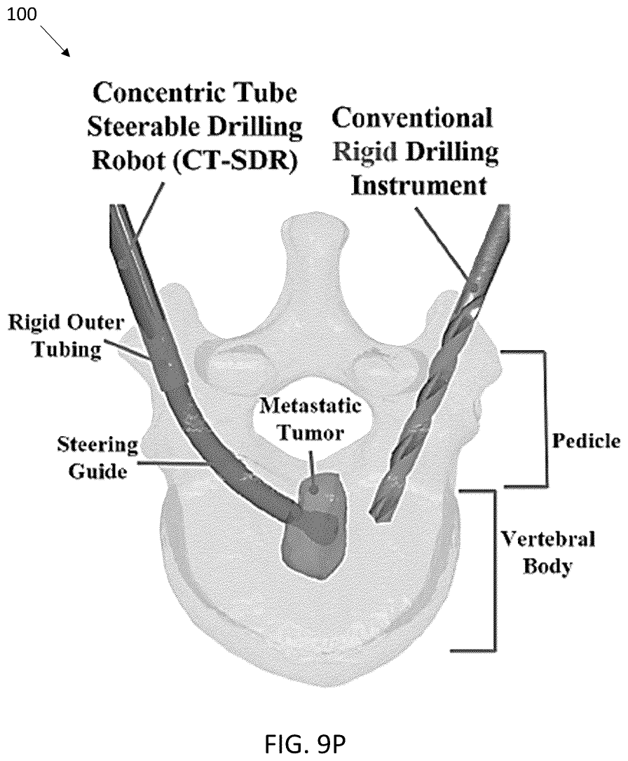

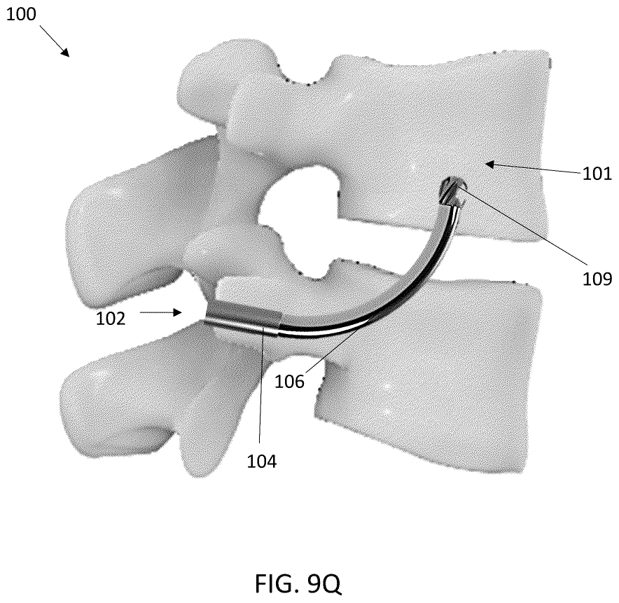

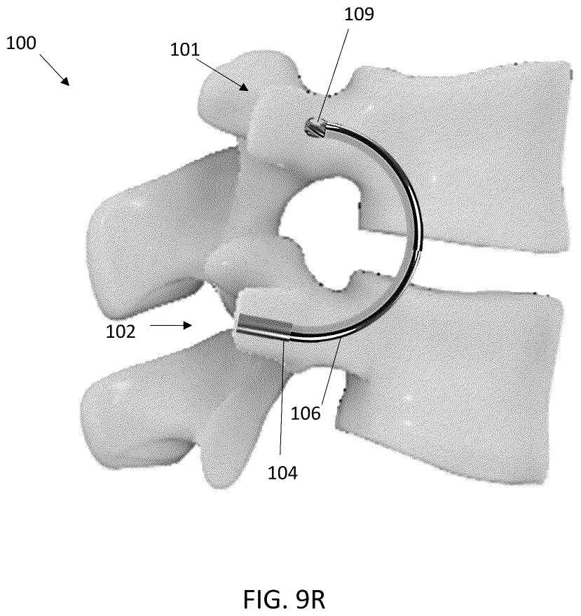

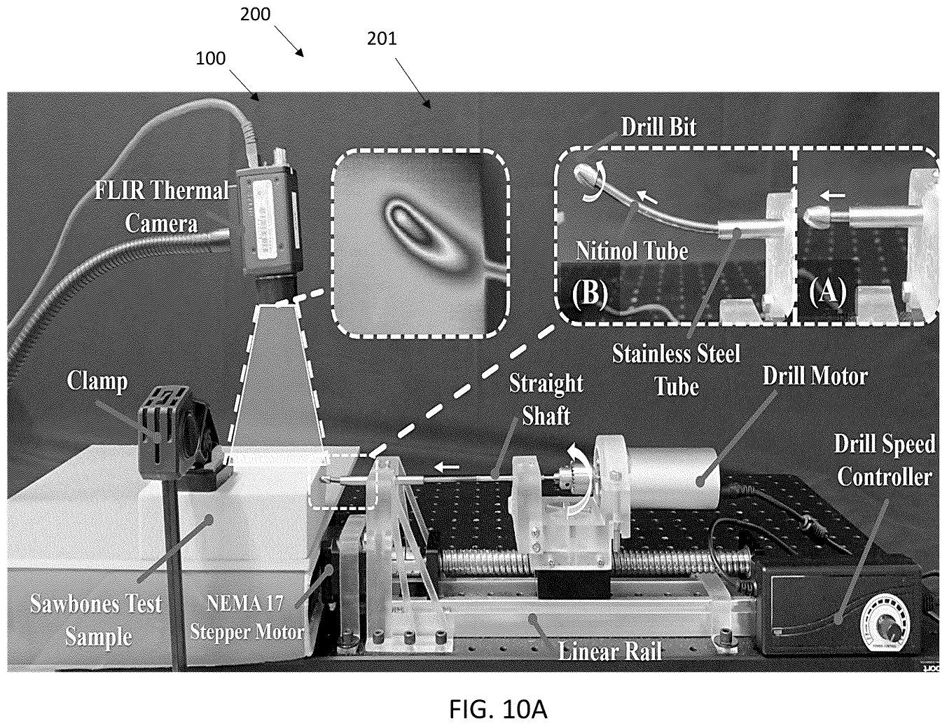

FIG. 8 and FIGS. 9A-9P show example applications of the device 100. FIG. 8 shows example drilling trajectories for the drilling robot device 100. In some embodiments, the device 100 can drill long curved trajectories inside bones with complex curved anatomies such as the pelvis and vertebrae, for example.

FIG. 9A shows a diagram depicting an example drilling robot device 100 in operation. The device 100 can be manufactured to follow a preset drilling trajectory to avoid obstacles and/or hit a target area. The obstacles and target area can be identified by the characterization of a bone tissue. In some embodiments, the obstacles can include osteoporotic bone, bone with low mineral density, nerves, and/or blood vessels, for example. In some embodiments, the target area can be a cancerous tumor.

FIG. 9B shows an example of a J-shaped drilling trajectory operation utilizing the drilling robot device 100 in a vertebra fixation procedure.

FIGS. 9C and 9D show an example of a minimally invasive cavity cutting drilling trajectory operation utilizing the drilling robot device 100 in a vertebra fixation procedure. The device 100 can be introduced to the target location using a linear or curved trajectory, and then can be rotated to create a cavity in a minimally invasive way. Furthermore, it can then continue drilling a specific trajectory.

FIG. 9E shows an example of a U-shaped drilling trajectory operation utilizing the drilling robot device 100 in a vertebra fixation procedure.

FIG. 9F shows an example of a multiple J-shaped branch drilling trajectory operation utilizing the drilling robot device 100 in a vertebra fixation procedure.

FIG. 9G shows an example of an S-shaped drilling trajectory operation utilizing the drilling robot device 100 in a vertebra fixation procedure. The example device 100 includes a first inner tube 106 and a second inner tube 106′ movably nested within the first inner tube 106.

FIG. 9H shows an example of long curved drilling trajectory operations utilizing the drilling robot device 100 in a pelvis fixation procedure.

FIG. 9J shows an example of a multi-segment drilling trajectory operation utilizing the drilling robot device 100 in a pelvis fixation procedure. The example device 100 includes a first inner tube 106, a second inner tube 106′ movably nested within the first inner tube 106, and a third inner tube 106″ movably nested within the second inner tube 106′.

FIG. 9K shows an example of a curved drilling trajectory operation utilizing the drilling robot device 100 for ACL reconstruction procedure in a knee.

FIG. 9L shows an example of a curved drilling trajectory operation utilizing the drilling robot device 100 for PCL reconstruction procedure in a knee.

FIG. 9M shows an example of a multi-branch curved drilling and cavity cutting trajectory operation utilizing the drilling robot device 100 in a femoral head osteonecrosis or fixation procedure.

FIG. 9N shows an example comparison of the drilling robot device 100 drilling a curved trajectory through the pedicle toward high BMD regions of an L3 vertebra compared with a conventional rigid drilling instrument constrained to linear trajectories. The figure also indicates the complex anatomy of vertebra together with the high and low BMD regions of vertebral body.

FIG. 9P shows an example comparison of the drilling robot device 100 drilling a curved trajectory through the pedicle toward a metastatic tumor of a vertebra compared with a conventional rigid drilling instrument constrained to linear trajectories.

FIGS. 9Q-9R show examples of drilling trajectories utilizing the drilling robot device 100 in vertebra fixation procedures. In some embodiments, the procedure would require a flexible fixation instrument to be placed through the drilled channel, such as a flexible pedicle screw for the J-shape trajectory and either a flexible screw or other flexible fixation device such as cables would be required for the u-shaped trajectory.

A manual handheld or robotic manipulation system (201 or 301) can be chosen based on the number of inner tubes 106 utilized and which abovementioned trajectory needs to be drilled. For simple J-shape trajectories or simple cavity drillings a single inner tube 106 is sufficient. However, for complex trajectories and cavity drilling such as S-shaped, a multi-segmented, and multiple branched trajectories, a robotic manipulation system (201 or 301) and a device 100 utilizing multiple inner tubes (106, 106′, 106″, . . . ) is necessary for more accurate drilling results. This is mainly because manual control of a device with multiple inner tubes (106, 106′, 106″, . . . ) is not intuitive and is very difficult.

Furthermore, the handheld and/or robotic manipulation system (201 or 301) can be attached to a secondary robotic system to perform a robot-assisted, tele-operation, image-guided, surgeon-in-the-loop, and/or fully autonomous surgical drilling procedure. For example, for the system 200 shown in FIG. 4, the operation can be performed by a surgeon holding the robot directly, such as current Medtronics or Zimmer Biomet robots, or via a tele-operated robot such as the Da Vinci robot, or via an autonomous robot.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention, and are not to be construed as limiting in any way the remainder of the disclosure.

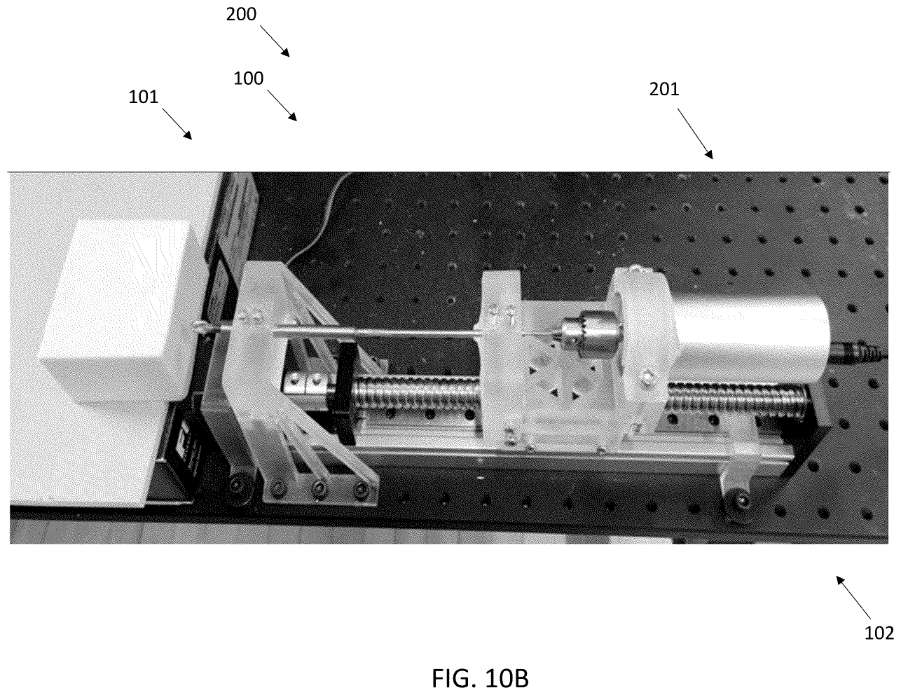

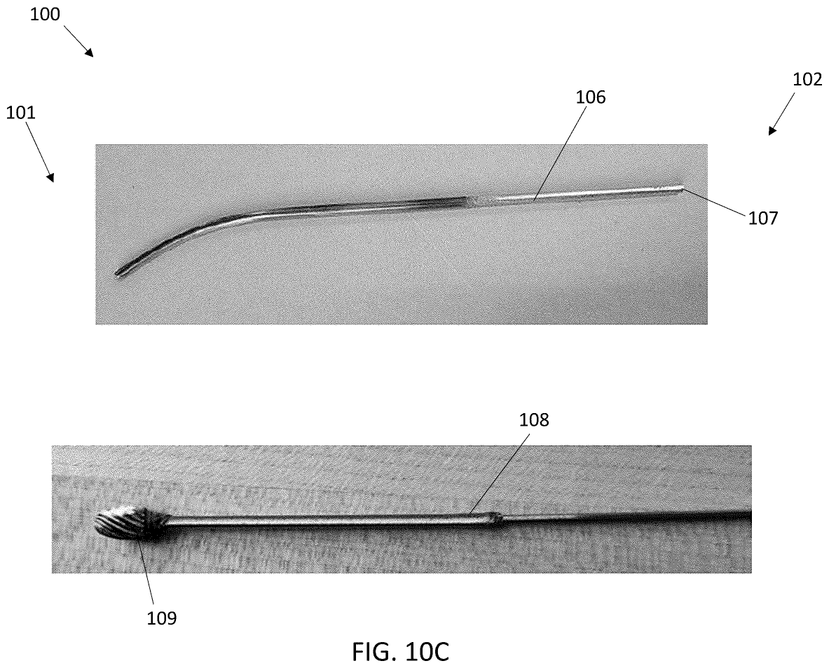

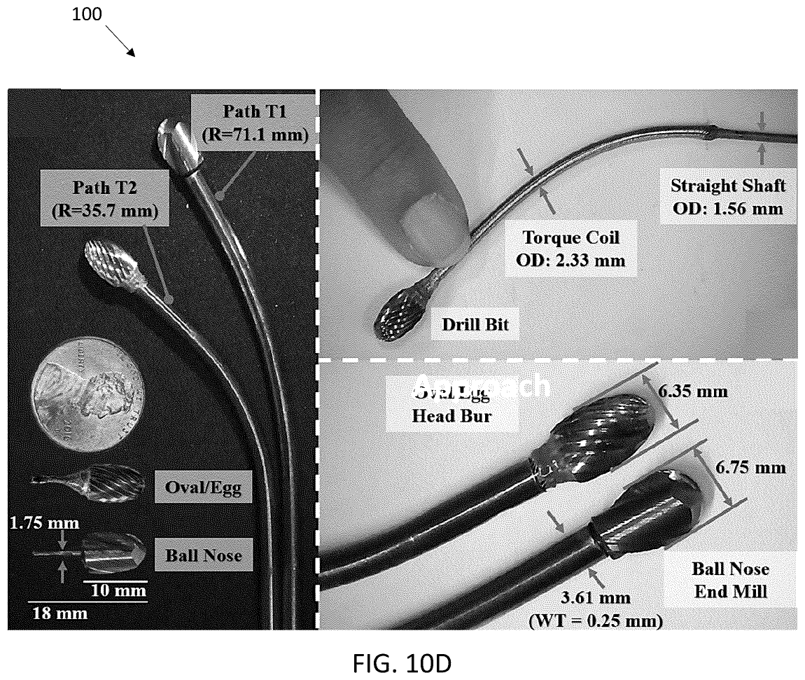

FIGS. 10A-10D show an example experimental setup of the drilling robot system 200 comprising the device 100 and manipulation system 201. The system 200 was mounted to a table and was setup to drill test curved trajectories into a Sawbones® block. A leadscrew system was used as the manipulation system 201.

In an operating room setting, a surgeon would align the surgical drill with the vertebra and slowly advance the drill in to cut the material and then back the mechanism straight out. This situation was replicated using the experimental setup shown in FIGS. 10A and 10B designed to validate the concept of utilizing the device 100 for spinal fixation procedures. To simulate human bone, a Sawbones® bio-mechanical bone model phantom (block 10 PCF, Pacific Research Laboratories, USA) was used (see F. Alambeigi et al., “A curved-drilling approach in core decompression of the femoral head osteonecrosis using a continuum manipulator,” IEEE Robotics and Automation Letters, vol. 2, no. 3, pp. 1480-1487, 2017). The device 100 was mounted to an optical breadboard with a test sample held in front of the system. To visualize the interior of the test sample during testing, a thermal camera (FLIR A65sc, Teledyne FLIR LLC) was set up perpendicular to the cutting plane. As the device 100 was actuated through the test sample, the heat generated would spread through the block and, as the drilling was near the surface of the block, it could be picked up by the thermal camera. This allowed for monitoring in real time and for a second avenue of analysis after the test's conclusion. An additional camera was set up separate from the other components to track overall cutting time and closely monitor externally the performed experiments.

As shown in FIGS. 10A and 10B, the example experimental actuation mechanism 111 comprised of one NEMA 17 stepper motor and linear stage with a linear ball screw (B085TG12D1, Amazon) to provide the insertion degree of freedom, a mini electric handheld drill (B075SZZN4J, Amazon) provided the required rotational speed and cutting torque for the flexible instrument, and 3D resin printed supports and mounts were also utilized. The stepper motor was controlled with an Arduino Uno R3 microcontroller board and a custom-written program controlling the linear speed of the motor. The rotational speed of the motor was also set using the provided controller of the mini handheld drill. The design of the actuation mechanism 111 was centered around the alignment of the tubes involved in the device 100 and the force required to push forward or retract the NiTi inner tube 106 inside the stainless steel out tube 104. To overcome the friction present between the concentric tubes and to provide the force required to bend the NiTi inner tubes 106 from their heat-treated shape, the device required a motor that provided the appropriate amount of torque without weighing down the system, and while maintaining a compact size. The NEMA 17 stepper motor not only allowed for a compact system but also for precise control during experimentation. The 3D printed parts added rigid supports to the system to resist internal friction forces, allowing for the continued alignment of the concentric tubes and creating a strong base for the motor to push against.

FIG. 10C shows exemplary experimental components of a drilling robot device 100 in accordance with some embodiments. As shown, an inner tube 106 of device 100 was differentially heat-treated. This provided a curved portion of the inner tube 106, while the untreated portion remained linear. Additionally, an example flexible drive shaft 108 with tool tip 109 is shown. The flexible drive shaft 108 comprises a torque coil and the tool tip 109 comprises a drill bit. In addition to the flexible drive shaft 108, other flexible instruments can be passed through the second concentric through hole 107 of the inner tube 106.

FIG. 10D shows additional exemplary experimental components of a drilling robot device 100 with exemplary dimensions. The radius of curvature for path T1 was 71.1 mm, and for path T2 was 35.7 mm. The figure also displays the drill bits prior to insertion into the torque coil. Both bits have a head length of 10 mm, an overall length of 18 mm, and a shank diameter from 1.5 mm to 2 mm when moving from tip towards the head. For the device 100 in this experimental example, a singular 70 mm, heat treated NiTi inner tube 106 was nested within a straight stainless steel outer tube 104 (89895K421, McMaster-Carr) with an identical length and 1.25 mm wall thickness. Compared to the typical concentric tube robots, the proposed device 100 uses a NiTi tube with larger diameter and wall thickness to provide more structural stiffness for the drilling system (i.e., outer diameter of OD=3.61 mm and a wall thickness of WT=0.25 mm) (Euroflex GmbH, Germany). As shown in FIG. 10D, after heat treatment, the tubes held the planned radii of curvature while maintaining the super-elastic properties originally sought after. To minimize the amount of NiTi tube used in the system and provide the adequate length for housing the flexible cutting tools, the NiTi tube was attached to a straight stainless-steel tube (89895K712, McMaster-Carr) with 80 mm length and 3.175 mm outer diameter. As shown in FIG. 10A, this stainless-steel tube was attached to the linear stage mechanism to advance and retract the NiTi inner tube 106.

As shown in FIG. 10D, each flexible cutting tool was comprised of a rounded tool tip 109 (drill bit, burr, or mill) at the distal end 101, a flexible drive shaft 108 (torque coil) in the middle, and a straight rigid shaft at the proximal end of the tool adhered to each other using epoxy (1813A243, McMasterCarr). To investigate the influence of the tool tip 109 geometry on the cutting performance of the device 100, two tool tips 109 were selected and tested during the experimentation step including a carbide oval/egg head bur (42955A35, McMaster-Carr) and a ball nose end mill (8878A42, McMaster-Carr). Of note, both of these geometries have the capability to cut not only in the forward direction but also with the sides of the cutting tool making them promising choices for a tool that needs to cut in non-linear trajectories (see F. Alambeigi et al., “A curved-drilling approach in core decompression of the femoral head osteonecrosis using a continuum manipulator,” IEEE Robotics and Automation Letters, vol. 2, no. 3, pp. 1480-1487, 2017). Both of these tools were ground down to have a shank diameter between 1.5 mm to 2 mm and given a slight taper to accommodate for the added flexible drive shaft 108 (torque coil). The heads of both the oval head and ball end mill were 10 mm in length, the shanks 8 mm in length, and had cutting tip diameters of 6.35 mm and 6.75 mm, respectfully. Both of these tools' geometries are shown in FIG. 10D.

The tool tip 109 at the distal end 101 and the rigid shaft at the proximal end 102 of the tool were separated by a flexible drive shaft 108 with 70 mm length (Asahi Intec. USA, Inc.), designed to be fed through the NiTi inner tube 106 and transmit the provided rotational torque by the motor of the actuation mechanism 111 to the tool tip 109. To not crush and/or damage the flexible drive shaft 108 and have a higher torque transmission efficiency, the straight rigid shaft was designed to be gripped by the chuck of the motor. For the oval head bur, the rigid shaft was a stainless-steel rod (888915K11, McMaster-Carr) with a diameter of 1.56 mm whereas the ball end mill had a brass tube (8859K231, McMaster-Carr), with the same diameter, allowing a material removal/agitation or cooling mechanism with water or air to be added to the cutting area through the inside of this innermost component if needed.

Heat treatment of the NiTi plays one of the largest roles in the manufacturing of the device 100. To program the desired drilling trajectory in the NiTi inner tube 106, the tube (Euroflex GmbH, Germany) was heat treated in a furnace while being constrained to the desired shape in a custom CNC fabricated stainless steel jig. The heat treatment instruction provided in previous studies (see D. Hodgson SMST-2000, Proc. Int. Conf. on Shape Memory and Superelastic Technologies, 2001, pp. 11-24) were followed to perform this process. The jigs had radii of curvature of 35 mm and 69.5 mm. These curvatures were arbitrarily chosen based on the paths that the tubes would take if placed through the pedicles of an L3 vertebra. Tubes with these curvatures would grant a surgeon access to areas within vertebral body that are not currently accessible with the existing rigid instruments.

Each experiment run with the device 100 was used to evaluate the system's performance in regards to the system requirements mentioned above. Particularly, drilling time and repeatability/predictability of the resulting drilling tunnel (i.e., hole curvature and diameter), the structural strength, buckling, and failure modes of the device 100 together with the cutting performance of the device 100 were thoroughly investigated. Several factors were determined to have potential effects on the system during initial experiments with the device 100, including the insertion speed of the inner tube 106, the rotational speed of the tool tip 109, and the tool tip 109 geometry.

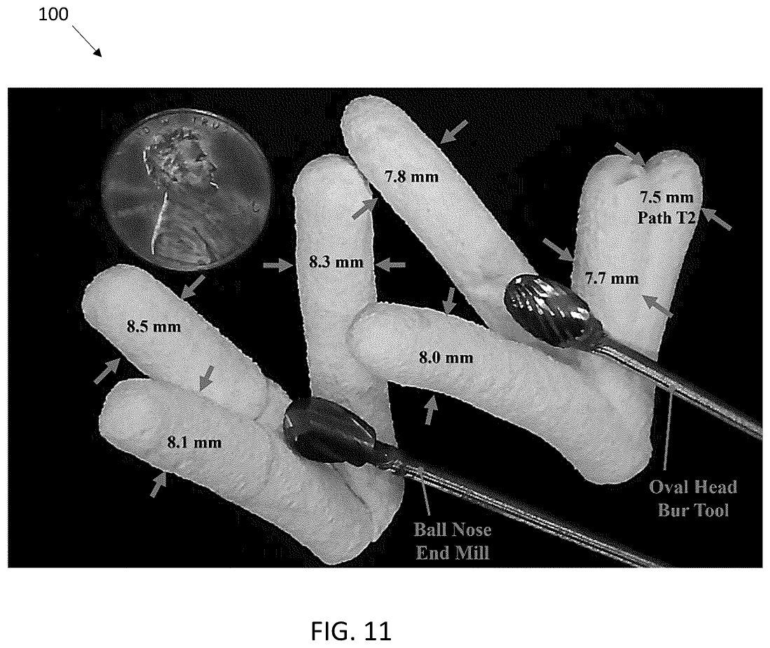

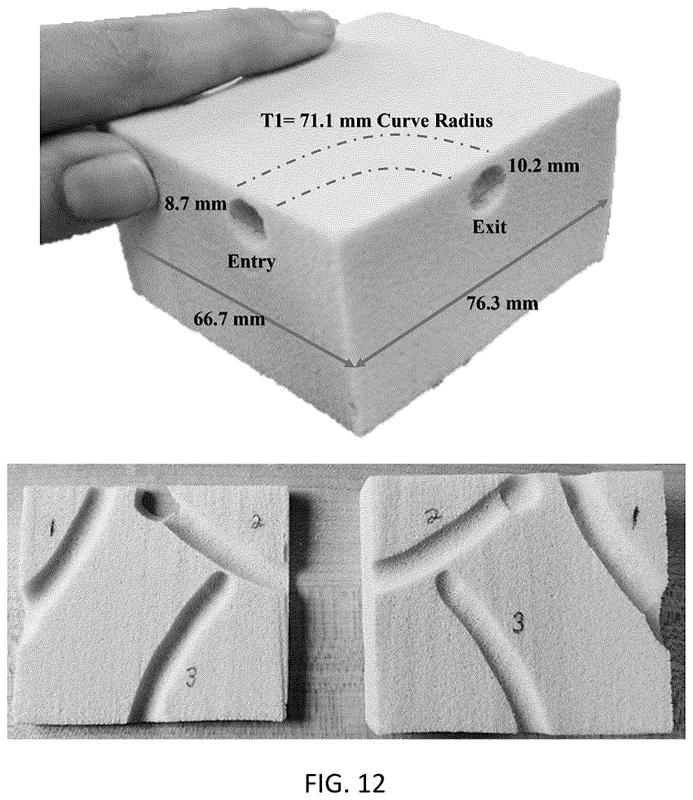

FIG. 11 shows 3D plaster models of the interior of experimental multi-branch drilling trajectories. The left model shows three J-shape trajectories cut with the ball end mill tool from a single entry point while the right model shows four J-shape trajectories cut with the oval head bur tool from a single entry point, three with the 71.1 mm radius NiTi tube and one with the 35.7 mm radius tube (Path T2). The experiment was conducted to determine the abilities of the device 100 in drilling multiple out-of-plane J-shape trajectories from a single access point. This feature enables reaching multiple locations within the vertebral body after entering through the same entry point, thus minimizing the extra unnecessary removal of the bone and weakening the structure of the vertebral body. This experiment was run with an insertion speed of 0.85 mm/s, rotational speed of 8250 rpm, and with both drilling tool tips 109 (drilling instruments). To compare the performance of the drilling instruments, both tools were used in the experiments, but a focus was placed on the 71.1 mm radius tube, to show longer path lengths. As shown in FIG. 10, two sets of experiments were performed, one with four and the other with three J-shape trajectories from single entry points. As the thermal camera used in previous experiments is unable to visualize the drilled tunnels that traverse deeper into the test sample and simple planar slices would be unable to fully visualize the drilled paths, the branching paths were filled with plaster (B08XW9ML9P, Amazon) and the Sawbones® of the test sample were treated as a mold. The Sawbones® material was then removed to reveal a full visualization of the interior paths. This 3D sample, shown in FIG. 11, allowed for accurate inspection of the radius of curvature, length, and smoothness of the drilled trajectories in the laboratory instead of using complex imaging modalities such as CT scans.

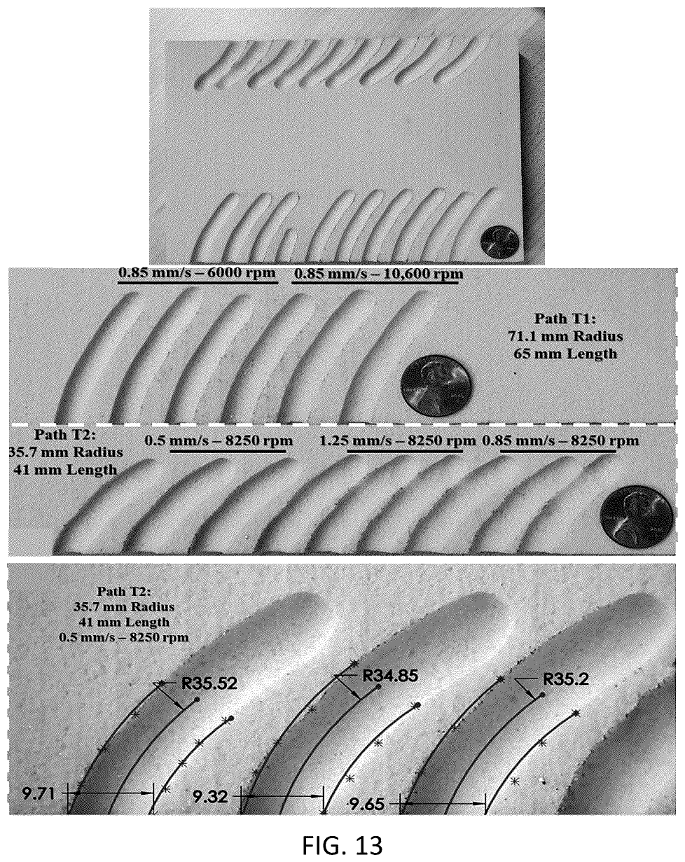

As shown in FIGS. 12 and 13, multiple curved trajectories were drilled into Sawbones® blocks to experimentally test various curvatures and the repeatability and accuracy of drilling the trajectories.

NiTi tubes heat treated for this project with each trial being repeated 3 times. A total of 30 tests were performed in all with the oval head drill tip. Of note, in these experiments, the bending plane of the inner tube 106 was fixed to be parallel with the optical table. After the main experiments were concluded, the ball nose end mill was also tested with both insertion speeds of 0.85 mm/s and 1.25 mm/s, and a drilling speed of 8250 rpm. The drilling started without a pilot hole or starting assistance of any kind and was simply advanced into the flat face of the test sample. This replicates the starting conditions of a vertebral insertion drilling in which, in real surgical scenarios, the surface is first flattened for easier insertion. In each trial, the device 100 was advanced the full length of the curve of the respective heat treated NiTi inner tube 106 used in that trial, and the thermal camera video and external video recorded.

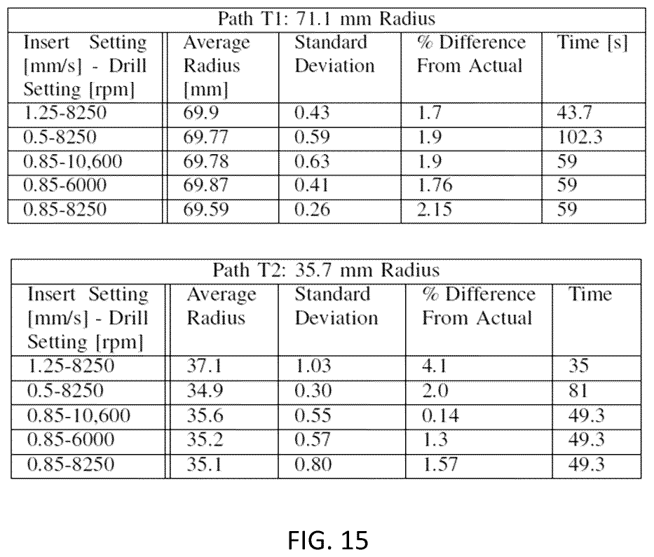

After the trials were completed, videos were taken of the interior of each trial using a 5.5 mm Borescope Camera (TELESION Inc., China) inserted through the drill's entrance hole before the test samples were cut open to assess the curvatures and internal smoothness of each pathway. Next, the samples were cut along the mid-line of the entrance holes and were closely inspected and photographed using a digital microscope (Jiusion, China). The images were then analyzed with a computer vision algorithm to determine each path's radius of curvature and compared to the ideal and the actual radii of the NiTi inner tubes 106. FIG. 13 displays the cross-sectional views of the repeated drilled trajectory T2 (with 35.7 mm NiTi cannula) and analyzed with the computer vision algorithm. Also, Table I and Table II of FIG. 15 summarize the experimental results of the performed experiments with respect to the drilling time and repeatability (i.e., drilled tunnel radius of curvature and diameter) of the performed trials with both NiTi cannulas and the cutting instruments.

FIG. 13 shows a cross sectional view of some of the drilling experiments including a view of Path T1 tests with different rotational drilling speeds while the insertion speed of the device 100 was held at a constant 0.85 mm/s, and a view of Path T2 tests with variable insertion speed while rotational drilling speed was held at a constant 8250 rpm. The lower right image depicts an analysis of the drilling trajectories (i.e., radius of curvature and diameters of the drilled trajectories) obtained with inner tube 106 (steering cannula) insertion speed of 0.5, 0.85, and 1.25 mm/s, and three rotational drill speeds of 6000, 8250, and 10,600 rpm. Five trials were run for each of the 35.7 mm radius NiTi curvature. All measurements were made in mm.

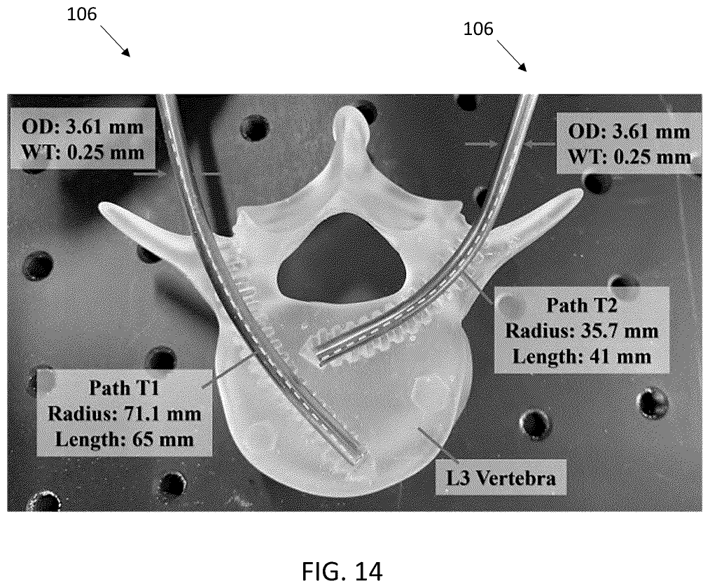

FIG. 14 shows examples of experimentally heat-treated inner tubes 106 of different curvatures over curved trajectories in a 3D printed L3 vertebra for verification that the inner tubes 106 can be heat-treated to follow a planned trajectory.

FIG. 15 shows tables of example experimental results for path T1 and path T2. The two main results were the time it took for the device 100 to progress through the entire path, and the repeatability of the drilled path. The time for the device 100 to drill the complete path began when the insertion of the device 100 started, right before it touched the sample piece, and ended when the device 100 was turned off and removed from the test sample. This test was to ensure that the addition of the device 100 to a surgeon's operational procedure would not add a significant additional amount of drilling time compared to currently used rigid instruments. As predicted by preliminary testing, and summarized in the top table of FIG. 15, the faster insertion times were generated from the fastest insertion speed of 1.25 mm/s with, on average, a full path drilled with the 71.1 mm radius NiTi inner tube 106 taking 43.7 seconds. As reported in bottom table of FIG. 15, the NiTi inner tube 106 with a 35.7 mm radius of curvature, took 35 seconds on average to fully drill a path through the Sawbones® samples. The smaller radius had a smaller path length which led to the difference in these two times, where the 71.1 mm radius had an arc length of 65 mm while the 35.7 mm radius had an arc length of 41 mm.

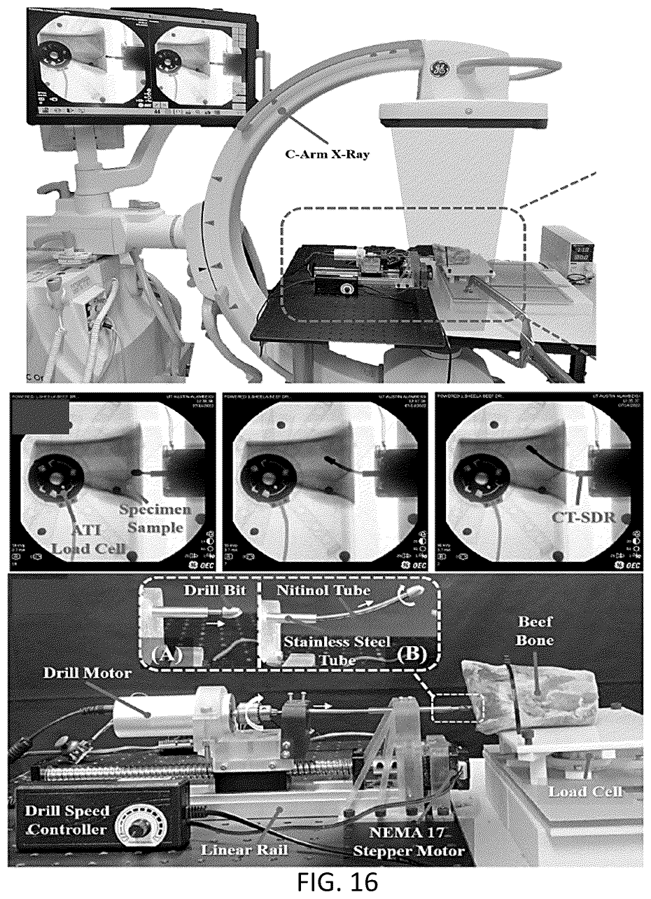

FIG. 16 shows an experimental set-up used to evaluate the performance of the system including a C-arm X-ray machine, a six-axis force/torque load cell, an animal bone test sample, and a holding mechanism. An overview of the full set-up is shown in at the top section with the C-arm's visual cone depicted. The bottom section gives a closer view of the system and the concentric tube actuation in the inset subfigures. The middle section displays the view from the C-arm at the beginning, middle, and end of the drilling procedure on the animal bone sample. This view also shows the utilized six axis force/torque load cell mounted under the holding mechanism.

The actuation unit shown include one NEMA 17 stepper motor and linear stage with a linear ball screw (B085TG12D1, Amazon) to provide the insertion DoF, a mini electric handheld drill (B075SZZN4J, Amazon) that provides the required rotational speed and cutting torque for the flexible instrument, and 3D resin printed supports and mounts. The stepper motor was controlled with an Arduino Uno R3 microcontroller board and a custom-written program controlling the linear speed of the motor. The rotational speed of the motor was also set using the provided controller of the mini handheld drill. The design of the actuation unit is centered around the alignment of the tubes involved in the system and the force required to push forward or retract the NiTi cannula inside the other stainless-steel tube. To overcome the friction present between the concentric tubes and to provide the force required to bend the NiTi tubes from their heat-treated shape, the system required a motor that can provide the appropriate amount of torque without weighing down the system and while maintaining a compact size. The NEMA 17 stepper motor not only allowed for a compact system but also for precise control during experimentation. The 3D printed parts added rigid supports to the system to resist internal friction forces, allowing for the continued alignment of the concentric tubes and creating a strong base for the motor to push against.

In an operating room setting, a surgeon would align the surgical drill with the vertebra and slowly advance the drill in to cut the material and then back the mechanism straight out, while monitoring the accuracy and safety of procedure using intermittent fluoroscopic images. This situation was replicated using the experimental setup shown in FIG. 16 designed to validate the concept of utilizing the system for spinal fixation procedures. In the first set of experiments, Sawbone biomechanical bone model phantoms (block 10 PCF, Pacific Research Laboratories, USA) were used to simulate human bone with medium osteoporosis before being replaced by animal bone samples in the later experiments (see A. Cetin and D. A. Bircan, “Experimental investigation of pull-out performance of pedicle screws at different polyurethane (pu) foam densities,” Proceedings of the Institution of Mechanical Engineers, Part H: Journal of Engineering in Medicine, vol. 235, no. 6, pp. 709-716, 2021). The system was mounted to an optical breadboard with the test sample held in front of the system. To visualize the interior of the test sample during testing, a C-arm X-ray machine (OEC One CFD, GE Healthcare) was set up perpendicular to the cutting plane. As the system was actuated through the test sample, the C-arm was able to show the location of the drill tip and steering cannula and cutting trajectory throughout the experiments. This allowed us to monitor and see the experiment in real time and for a second avenue of analysis after the test's conclusion. As shown, to measure the drilling forces imparted on the test specimen by the system during each test, a six DOF force/torque load cell (Mini45, ATI Industrial Automation) was also added to the set-up and sample holder. An additional camera was also set up separate from the other components to track overall cutting time and closely monitor, externally, the performed experiments.

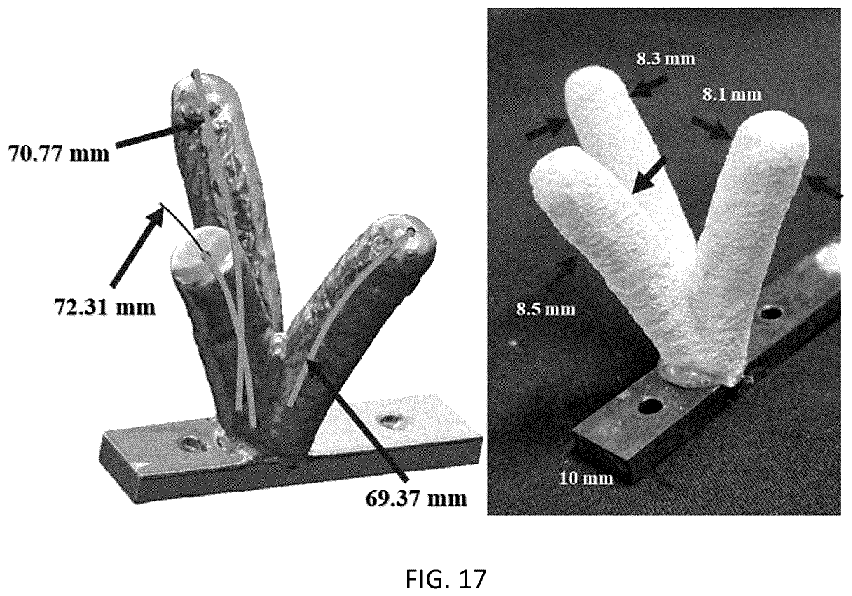

FIG. 17 shows plaster (right) and laser scanned (left) representations and measurements of the drilled out-of-plane Branch J-shape drilling tunnels. The plaster model was attached to a reference holder with a known width so accurate measurements could be taken of the path trajectories. The center of cross sections were used to measure the radius of curvature for the entire path. The three J-shape trajectories shown were drilled with the ball end mill tool from a single entry point with the 71.1 mm radius NiTi tube. Radius of curvature of the drilling trajectories and the diameters of the branches are shown in mm.

In addition to the performed single J-shape planar drilling trajectories, experiments were conducted to determine the abilities of the system in drilling multiple out-of-plane J-shape trajectories from a single access point. This feature enables reaching multiple locations within the vertebral body after entering through the same entry point thus minimizing the extra unnecessary removal of bone and weakening the structure of the vertebral body. Based on the results obtained in the performed J-shape drilling experiments, this experiment was run with an insertion speed of 0.85 mm/s, rotational speed of 8250 rpm, and with both drilling instruments. To compare the performance of the drilling instruments, both tools were used in the experiments, and a focus was placed on the 71.1 mm radius tube, to show longer path lengths. As shown in FIG. 17, a branch cutting experiment was successfully performed with three J-shape trajectories from single entry points.

As the C-arm used in the previous experiments provides 2D images and is unable to create clear and succinct visualization of the 3D directions/depths, the branching paths were filled with plaster (B08XW9ML9P, Amazon) and the Sawbones of the test sample were treated as a mold. The Sawbones material was then removed to reveal a full visualization of the interior paths. This 3D sample, shown in FIG. 17, allowed a qualitative inspection of the radius of curvature, length and smoothness of the drilled trajectories in the laboratory instead of using complex imaging modalities such as CT scans. Additionally, to quantitatively evaluate the performance of the system in branch cutting experiment, the plaster was then laser scanned using a handheld and portable 3D scanner (Artec Space Spider, Artec 3D Inc.) and its model exported into 3D CAD software (SolidWorks, Dassault Systemes) for further analysis. FIG. 17 displays the 3D rendering of the scanned plaster and the performed measured dimensions.

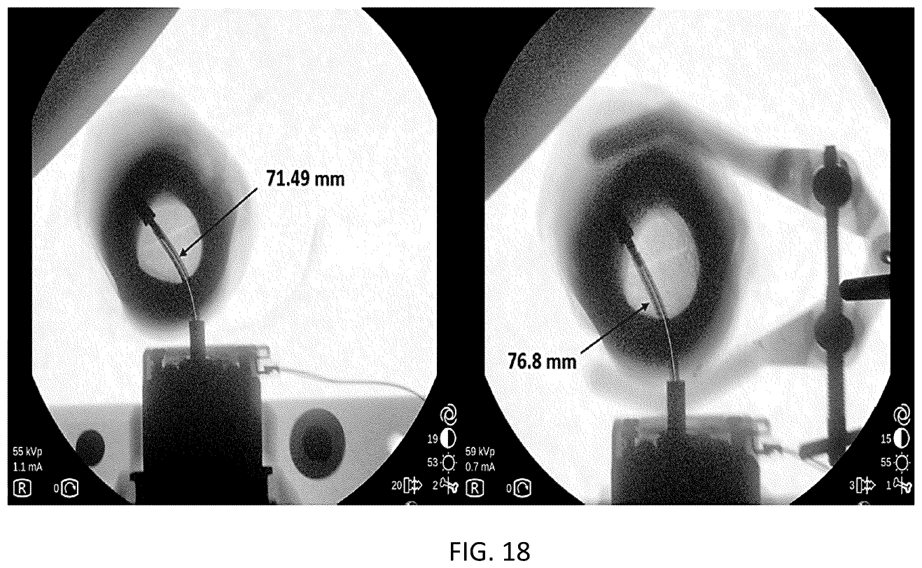

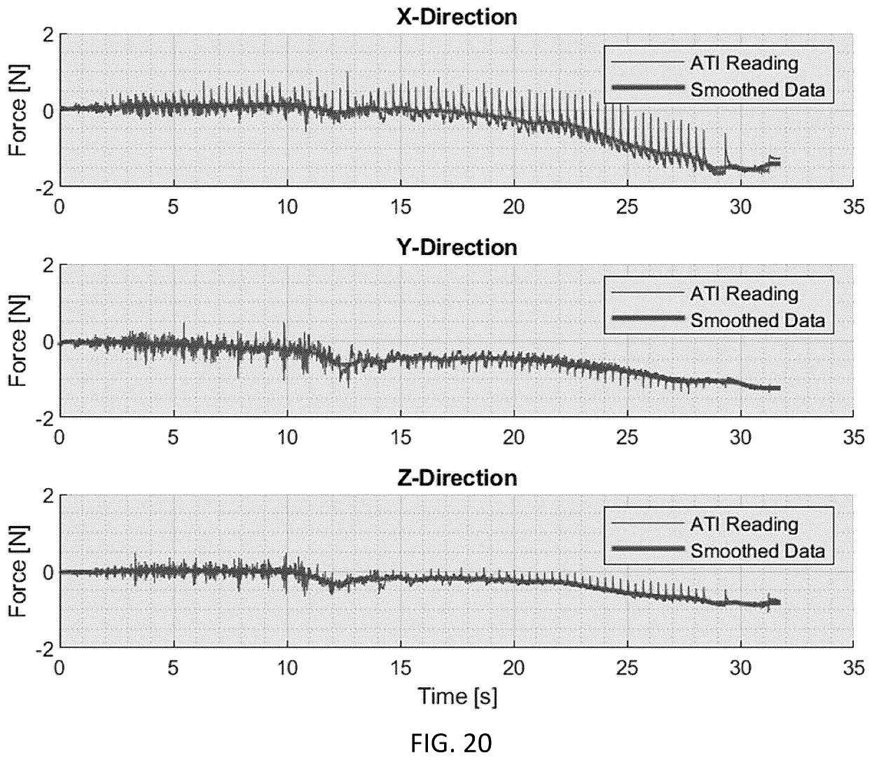

To evaluate the system in a more realistic drilling environment, its functionality was verified on animal bones using the setup shown in FIG. 16. For these experiments beef shanks of various sizes were used to show the curvature of the drill underneath the C-arm machine. Of note, due to the size of the bone samples and available drilling space, the 71.1 mm radius steering cannula was used. For each test, the system was aligned to drill across the horizontal plane for optimal viewing by the X-ray, the specimen was secured to the six-axis force/torque load cell supported platform, and the drill actuated through the specimen using the 0.85 mm/s insertion speed and 8250 rpm drilling speed. To confirm the drill was behaving as expected, the C-arm was used to capture live drilling trajectories throughout the drilling process as shown in FIG. 18. Moreover, FIG. 20 represents the recorded drilling forces for the test performed using the 71.1 mm radius steering cannula on the bone sample.

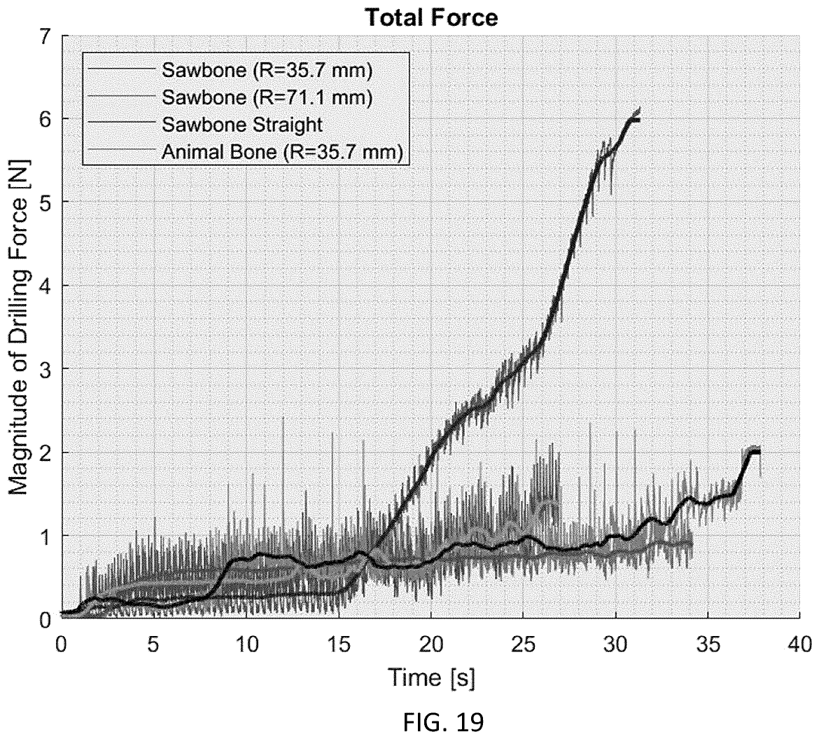

FIG. 19 shows Average magnitude of the drilling force throughout the drilling procedure on both Sawbone and animal bone samples captured by the force/torque load cell. The plot demonstrates the measured forces during drilling in a straight trajectory and two curved trajectories represented by R=35.7 mm and R=71.1 mm radii of curvature.

Further, FIG. 19 illustrates the average magnitude of the drilling forces in three repeated trials captured by the force/torque load cell (with frequency of 1 kHz) throughout the experiments performed on both Sawbone and animal bone samples. Also, this figure shows both measured and smoothed averaged forces using the smooth function in MATLAB (MATLAB, MathWorks) with a span of 100 during drilling in a straight trajectory and two curved trajectories represented by R=35.7 mm and R=71.1 mm radii of curvature. As can be observed, the magnitude of the measured drilling forces depends on the curvature of the drilling trajectory and the hardness of the drilling material. For the straight and R=71.1 mm drilled trajectory in Sawbone samples, the drilling force through the experiment is almost below 1 N whereas this value is dramatically increased (with maximum 7 N) for the drilled trajectory with the higher curvature (i.e., R=35.7 mm) in Sawbone samples. The drilling forces for the animal bone sample with R=71.1 mm is also higher than the straight (i.e., maximum 2 N) and similar to the test performed inside the Sawbone samples. FIG. 20 also shows the components of the measured and smoothed forces during animal bone drilling performed with the 71.1 mm radius of curvature. Of note, the measured drilling forces (both components and magnitude) are well within the measured ranges reported in the literature.

The results successful demonstrated the outperformance of the system as compared with the existing flexible drilling devices in the literature by repeatedly and consistently creating single and multiple J-shape drilling trajectories under 60 seconds with an error between 0.14-4.1% with respect to the planned trajectories. Additionally, when drilling through animal bone specimens, similar to the experiments performed on Sawbone samples, the system could recreate the planned curved trajectories while creating drilling forces that were well within acceptable ranges. Furthermore, the results indicate drilling times as low as 35 seconds for trajectories with 41 mm length and remarkable steering accuracy with maximum 2% deviation error from the planned trajectory.

To meet the needs of surgeons for complete treatment of spinal tumors, the system needs to provide required DoFs to enable a planar and out-of-plane generic J- and U-shape drilling trajectories as well as enabling cavity cutting based on the geometry of the tumor, flexible power transmission from a high rpm drill motor to carry rotational motion to the drill's cutting tip; sufficiently strong and flexible guides to steer the drill's cutting tip towards the areas of interest within the patient without deviation; and an actuation unit and control system to allow a surgeon to actively control the drill tip's position throughout a surgical procedure. The following sections will address in detail these different requirements and the components that were designed and manufactured to meet them.

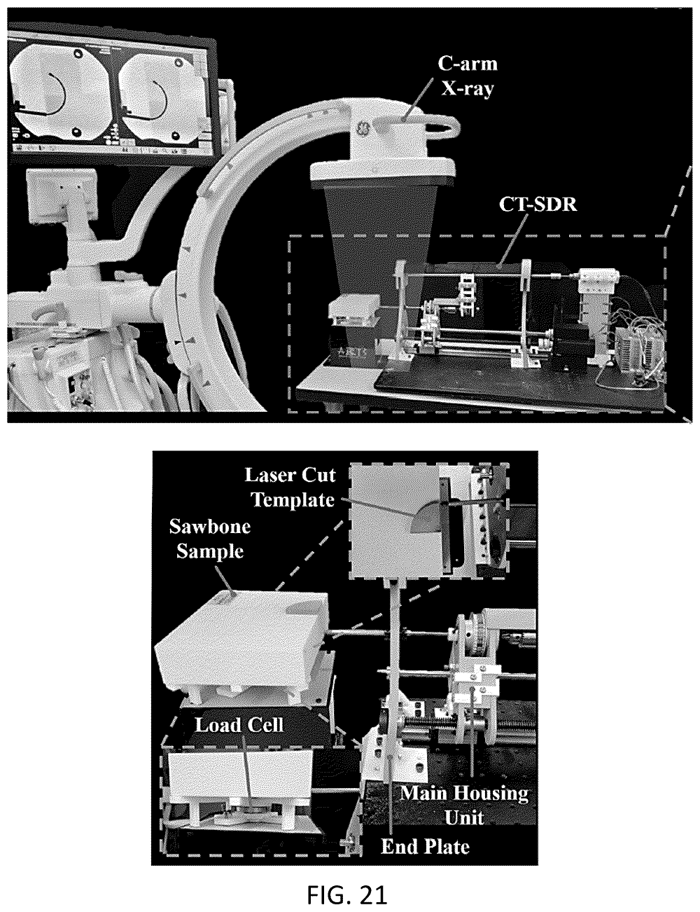

FIG. 21 shows an experimental set-up used to evaluate the system, including a C-arm X-ray machine, a six DoF load cell, laser cut template and Sawbone test sample. The top section shows an overview of the entire set-up with the C-arm's visual cone. The bottom section shows a closer view of the sample set up, and system, including a side view of the load cell, and a top view of the alignment of the laser guide with the system. The main housing unit of the system can also be seen in this view.

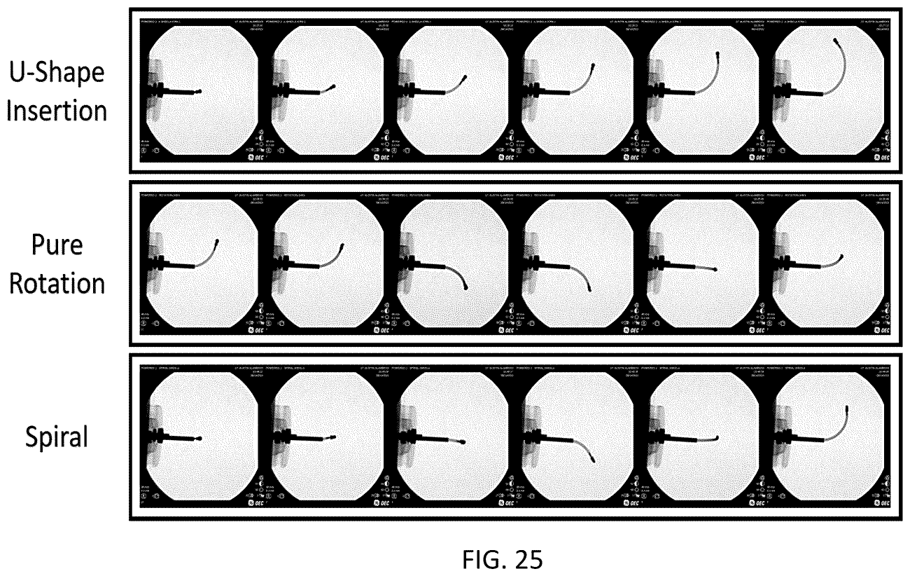

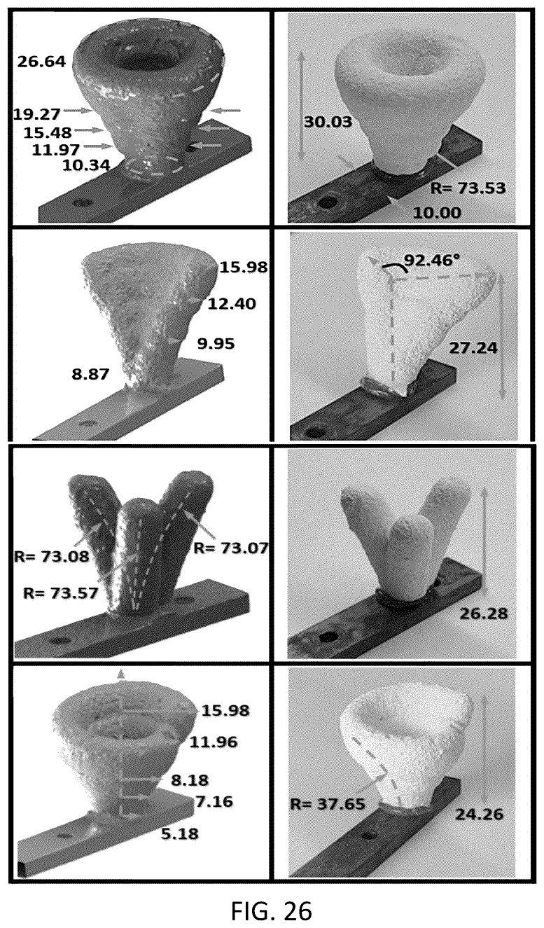

The experimental setup shown was used to thoroughly evaluate the performance of developed system in drilling planar and out-of-plane J- and U-shape trajectories together with creating cavities within a hard tissue. The experiments used Sawbone biomechanical bone model phantoms (block 5 and 10 PCF, Pacific Research Laboratories, USA) to simulate diseased human bones with lower bone mineral densities compared with a healthy tissue (see A. Cetin and D. A. Bircan, “Experimental investigation of pull-out performance of pedicle screws at different polyurethane (pu) foam densities,” Proceedings of the Institution of Mechanical Engineers, Part H: Journal of Engineering in Medicine, vol. 235, no. 6, pp. 709-716, 2021). As can be seen in FIG. 21, the system, mounted on an optical breadboard, was placed on a wooden table with the specimen held in front of the drilling tip on an acrylic stand. The materials of the table and the stand were selected so that they showed minimal interference with the C-arm X-ray (OEC One CFD, GE Healthcare) placed next to the system to monitor the system's progress through the test sample during experiments. The addition of the C-arm allowed for real time monitoring of the experiment by the user, and as an option for analysis after a test's conclusion. Views from the C-arm for different drilling experiments can be seen in FIG. 25. To measure and compare trends in drilling forces on the system's drill tip to previous studies, a six DOF force/torque load cell (Mini45, ATI Industrial Automation) was placed with a sample holder beneath the test specimen. An additional camera was placed separately from the other components to provide another viewpoint of the tests, and provide tracking for the experiments.

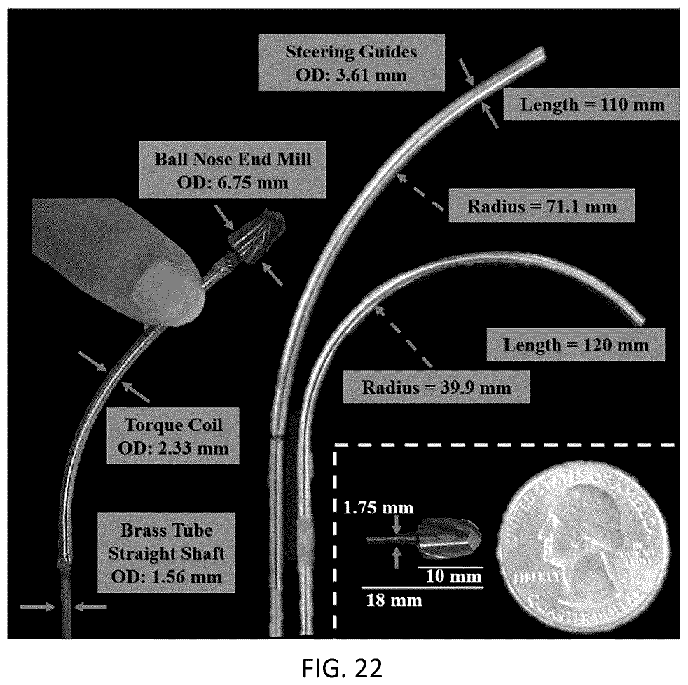

FIG. 22 shows the NiTi steering guides and the flexible shaft used in the experiment that correspond to the two inner tube systems of the system. A close view of the designed drill bit is shown in the subfigure. To create the required pathways and cavities, the system required steering guides that could move the drill's cutting tip into areas of interest by the surgeon. These guides would need to be both flexible enough to bend outward from the drill's entry point to access hard-to-reach areas, but strong enough to not deflect under the forces experienced by the cutting tip during drilling. The design takes advantage of the superelastic properties of NiTi metal (Euroflex GmbH, Germany), to provide a solution to these contrasting requirements. This superelastic, biocompatible, shape memory alloy is heat treated to a pre-designed curvature, which establishes the system's range of motion (see M. Drexel et al., The effects of cold work and heat treatment on the properties of nitinol wire, 2007, vol. 42665). Following heat treatment procedures the NiTi tubes in their original straight state were constrained to a desired shape using a CNC-fabricated stainless steel jig and placed in a furnace to create the designed drill trajectories (see D. Hodgson, “Fabrication, heat treatment and joining of nitinol components,” in SMST-2000, Proc. Int. Conf. on Shape Memory and Superelastic Technologies, 2001, pp. 11-24). After heat treatment, the tubes had curvatures of 71.1 and 39.9 mm radii. The steering guides used in the experiment are shown in FIG. 22. Of note the selected curvatures and tubing dimensions were arbitrarily chosen based on the geometry of an L4 vertebra. Nevertheless, these curvatures can readily be changed depending on the vertebral level and geometry.

When assembled into the system, the NiTi steering guide is nested within a larger stainless steel tube which provides the structural strength and rigidity required to constrain the NiTi tube into a straight configuration. The stainless-steel tube, which holds the role of the concentric tube's outer tube, is static in this design of the device, and the NiTi steering guide is actuated through it. As the guide is moved forward and out of the constraining outer tube, the portion of the guide removed from the stainless-steel returns to its heat treated, pre-programmed shape/curvature. In the process of returning to its pre-programmed shape, the guide steers the drill's cutting tip along the guide's trajectory, creating a curved and smooth drilled path.

As shown in FIGS. 6A-6C, each flexible tool comprises of a small rigid cutting tip, a flexible torque coil, and a straight rigid tube. To secure the components to one another, epoxy (1813A243, McMaster-Carr) is used at the part intersections. A ball nose end mill (8878A42, McMaster-Carr) was selected for the drill tip, as it produced clean smooth tunnels in earlier testing and has large flutes for faster material removal. Notably, the main concern in selecting a drill tip was the cutter's ability to remove material not only at the distal tip of but also on the sides of the cutter during planar and out-of-plane drilling procedures. The cutting tip has a diameter of 6.75 mm, a cutting tip 10 mm in length, and a shank 8 mm in length with a ground down diameter to 1.75 mm. The drill tip geometries and torque coil connection are shown in FIG. 22. The power transmission and the tool's flexibility was possible through the utilization of a torque coil (Asahi Intec. USA, Inc.) placed behind the drill's cutting tip. This torque coil is 115 mm in length, runs through the curved section of the NiTi tubing to serve as a method for delivering rotational motion around a curve in a reliable way. The coil did not connect directly to the drill chuck in the system's design to avoid crush damage to the coil, and instead was attached to a straight brass tube (8859K231, McMaster-Carr), with a diameter of 1.56 mm.

A primary requirement for the design of the two DoF device, as depicted in FIGS. 6A-6C, was to keep the utilized motors that actuate the steering guides and the cutting tool stationary to minimize the inertia of the system's moving components and subsequently the required power for actuating the system. To satisfy this design requirement, different methods were utilized for transmitting both translational and rotational motions through the system, to produce the desired motion for the steering guides and the system's drill tip. As shown in FIGS. 6A-6C, the system's insertion DoF is controlled by a NEMA 23 stepper motor (6627T530, McMaster-Carr) rotating a lead screw (98940A305, McMaster-Carr) to adjust the position of a nut (6350K41, McMaster-Carr) rigidly held within the main housing unit. The main housing is supported by a carriage sliding on a linear rail (6709K431, McMasterCarr), which allows for lower friction during translation as the lead screw actuates the housing. The NiTi steering guide's rotational orientation DoF is also controlled by a NEMA 23 stepper motor, this time controlling a spline shaft (61145K145, McMaster-Carr) which allows for the housing to have unrestricted motion along the linear rail, while still transmitting the rotational position of the connected stepper motor. The ball spline (61145K430, McMaster-Carr), within the main housing, is secured within a belt and pulley system connected to the NiTi steering guide's coupler. The designed pulleys were selected to have a 1:1 ratio for easy control by the stepper motor. An idler pulley was also designed into the system to ensure enough belt tension is maintained in the system.

To control the drill tip's rotational speed, a carriage was rigidly attached to the top of the main housing unit to serve as a channel for the high-speed rotations of the drill motor to be transmitted through. The drill motor (B075SZZN4J, Amazon) is mounted in a custom holder above the stepper motors at the back of the system and connected to another spline shaft (61145K143, McMaster-Carr) that runs the length of the system. Similar to the steering guide's rotational control shaft, this one allows the carriages to slide freely along the shaft direction while transmitting the rotational motion provided by the drill motor. Another 1:1 pulley system with idler pulley transmits this motion to a drill chuck (2812A19, McMaster-Carr) mounted on a stainless-steel hollow shaft. Also, as shown in FIGS. 6A-6C, the NiTi steering guide is attached to the main housing unit with a designed 3D printed coupler and set screw, which allows for the main housing components to control the guide's position and orientation. End plates placed at either end of the system provide support for many of the actuation unit's moving parts. These plates and the stepper motor mounts were 3D printed in PLA and secured to an optical breadboard for stability.

The utilized stepper motors were controlled with Rtelligent R60 motor drivers (B07SBFZ596, Amazon), an Arduino Uno R3 microcontroller board, and a custom program written with the AccelStepper.h Arduino library. The written program allowed for independent control of both the insertion and rotation degrees of freedom, or could be modified to control these freedoms simultaneously. The speed of the system in both rotation and insertion were also adjustable allowing us to optimize the different settings used in drilling.

Each experiment run with the system was designed to test the capabilities of the proposed two DoF robotic system and evaluate if it could reliably produce predictable long planar and out-of-plane curved drilling trajectories and minimally invasively remove cavities of material by entering from a small hole (e.g., vertebrae's pedicle) with the diameter of the drill bit and remove materials within the anatomy (i.e., vertebral body).

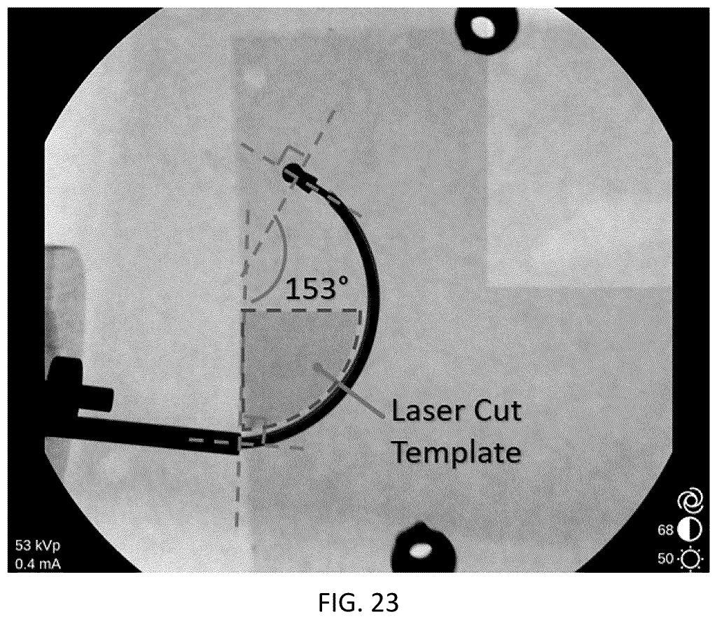

FIG. 23 shows an X-ray view of a U-shape trajectory test performed with a 39.9 mm steering guide in PCF 10 Sawbone. Visible at the top is a 35 mm radius laser cut template to view the accuracy of the system's path. U-shape drilling is an extension and extreme representation of the J-shape planar drilling concept, in which a NiTi steering guide is inserted and held by the system with the cutting plane parallel to the optical table's surface. However, the steering guides used in U-shape drilling are much longer and take the drill tip through a nearly 180 rotation as the drill tip is actuated through a circular trajectory. For this test, a 39.9 mm radius steering guide with a length of 120 mm was used. A 10 PCF Sawbone test sample was secured with the front face of the sample perpendicular to the system's initial cutting direction. The drill motor was accelerated to 8250 rpm, which in turn rotates the system's cutting tip at the same speed. Once the drill tip was at the desired cutting speed, the drill was actuated forward at 1.6 mm/s for the length of the steering guide. When the steering guide reached the end of its length, the drill motor was powered off the C-arm was used to take X-ray images of the system's tip position within the sample, and a laser cut template corresponding to an ideal 35 mm radius steering guide was used to determine the accuracy of the drilled U-shape trajectory. The angle of the final cut was measured from the analyzing the angle between the insertion orientation of the system's drill tip and the final orientation. This could be measured via the X-ray images taken of the test. FIG. 23 shows the X-ray view of the drilled U-shape trajectory and the used laser cut template to evaluate the accuracy of the system. Also, FIG. 25 shows the X-ray images demonstrating progression of the system through the Sawbone samples during this experiment.

The original design of the system was centered around its ability to produce out-of-plane cuts both through multiple J-shape branch trajectories and through simultaneous rotations and insertions while within a test sample. Several variations of cavity drilling tests were performed with both the 39.9 mm and 71.1 mm steering guides to evaluate the success of the system's design. In each test, the Sawbone sample was secured in front of the system, the drill motor was accelerated to 8250 rpm, and the test was carried out with an insertion speed of 1.6 mm/s and a rotation speed of 9.6°/s (unless otherwise specified). FIG. 25 shows the X-ray images demonstrating progression of the system through the Sawbone samples during the cavity drilling scenarios. The X-ray images show progression of a test by moving the device through free space with a 39.9 mm radius steering guide. The top section shows a U-shape trajectory view. The middle section shows a singular rotation of a pure rotation test, in which the system would do a pure rotation at several depths of cut. The bottom section shows a spiral test in which the rotation and insertion DoFs move together.

To test the initial functionality of the system's rotational DoF, a J-shape branch test was designed. In this test, the NiTi steering guide was actuated through the test sample in a J-shape trajectory, retracted fully, rotated out of plane, and re-inserted through the same entry point to drill another J-shape trajectory. This insertion/retraction/rotation was repeated until 3 paths had been drilled from the same entrance hole.

In an independent 2-DoF Drilling test utilizing pure rotational motion the system was tested with both insertion and rotation DoFs of the steering guides while the system's drill tip was within the test specimen. For this test, the system was inserted approximately 10 mm and then the insertion was paused as the system performed a full rotation of the steering guide before inserting and rotating again. This process was repeated for the length of the steering guide used for the test.

The final cavity tests conducted with the system were centered around coupling the insertion and rotation DoFs together. For this test, the system was set to run with an insertion speed of 0.96 mm/s and a rotation speed of 4.7°/s, chosen to ensure that a full rotation would occur before the system had translated a full length of the drill tip.

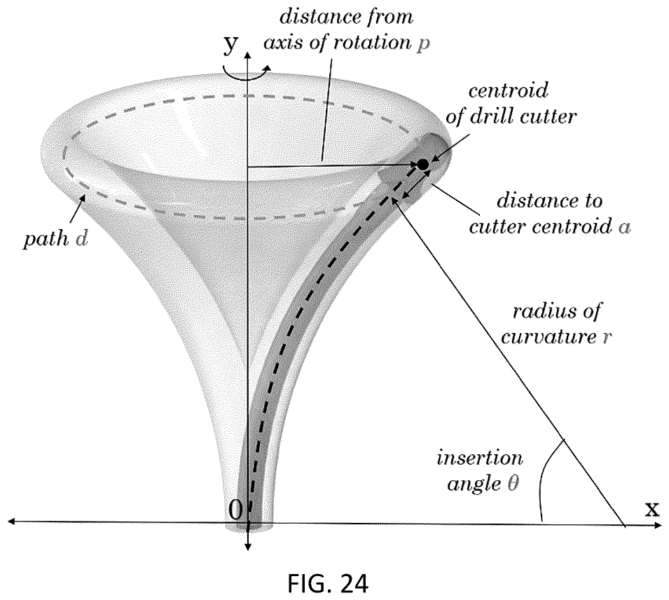

FIG. 24 shows a theoretical representation of the ideal cavity volume removed by the system. To theoretically calculate the volume of the drilled cavities and compare it with the experimental results, the second theorem of Pappus was used. As shown in FIG. 24, based on this theorem, the cutting volume caused by out-of-plane rotation of the drill bit about the horizontal axis of the stainless-steel tube and at each sequential insertion length of the NiTi tube can be calculated as follows:

$${V}_{i} ( s ) = A * d ( s ) \qquad ( 1 )$$

in which V is the volume of the cut ring at a given insertion step, A is the cross section of the drill bit, and d(s) is the distance around a full revolution for the centroid of the drill bit at a given insertion step. As shown in FIG. 24, d(s) can be calculated from the distance between the axis of rotation and the centroid of the drill bit, p:

$$d ( s ) = 2 * π * p \qquad ( 2 )$$

$$p = ( r - r * cos ( θ ) ) + a * sin ( θ ) \qquad ( 3 )$$

$$θ = \frac{s}{r} \qquad ( 4 )$$

where s represents the arclength, r is the radius of curvature, and θ represents the known pre-curved angle of the NiTi tube. A total volume of the removed cavity is then calculated from the summation of the cavity volumes Vi at each insertion distance and revolution of drill bit throughout a test.

After the conclusion of each test, as the resulting cavities were not clearly visible with a C-arm, 3D models were made from the drilled test samples. Plaster was poured through the entrance hole of the test and allowed to harden. The Sawbone material was then removed to leave an inverted view of the drilled cavity. These models were then laser scanned (Space Spider, Artec3D) and imported into 3D CAD software (SolidWorks, Dassault Systems) where they could be measured and analyzed. FIG. 26 shows the exemplary plaster and laser scanned models.

FIG. 23 shows the results of the U-shape drilling with a NiTi steering guide with a radius of curvature of 35 mm. From the figure, it is clear that the system can drill around an obstacle and reach a point 82 mm in a perpendicular direction to the entry trajectory. In this experiment, the angle of change in which the system's cutting tip has moved through during the test (as measured counter-clockwise from its original position) was 153°.

FIG. 26 shows 3D renderings (left) of actual cavity drilling models (right). The top section shows a pure rotational test performed in 10 PCF Sawbone with the 71.1 mm steering guide. The top-middle section shows a pure rotational test performed in 5 PCF Sawbone with the 39.9 mm steering guide, though rotated only 92° instead of a full 360°. The bottom-middle section shows a branches test in 10 PCF Sawbone with the 71.1 mm steering guide. The bottom section shows a spiral test performed in 5 PCF Sawbone with the 39.9 mm steering guide.

In the branches tests, the goal was to reach locations out of plane with accurate trajectories created by the steering guides. In a block of 5 PCF Sawbone, the steering guide with a radius of 71.1 mm produced 3 branches with an average radius only 2.6% different from the guide showing that out of plane cuts had little to no effect on the system's behavior.

Perhaps the most significant test run by the system, the addition of actuating the rotational DOF during the drilling process created the first true cavities seen by this system. As shown in the top section, the laser scanned render of the cavity rotation test run in 10 PCF Sawbone with the 71.7 mm steering guide. From the first rotation within the material to the final ring made by the drill tip, the diameter of the cut material more than doubled, going from 10.34 mm to 26.64 mm in a distance of 30.03 mm. The rotational tests took approximately 2 minutes. Tests were also run with a steering guide of radius 39.9 mm, instead of rotating a full 360° at each insertion step, the device was rotated 92.46° to create a partial cavity in 5 PCF Sawbone shown in the top middle section. The diameter of the projected cut increased from 17.74 mm to 31.96 mm.

The coupled insertion/rotation cavity tests displayed similar success to their decoupled counterparts. Drilling through 5 PCF Sawbone, the 39.9 mm radii steering guide was used while simultaneously actuating the rotational and translational components together. The result was a cavity similar in size and dimension to those in previous tests, with an insertion radius of 5.18 mm and a final radius of 15.98 mm as measured as the distance from the final drilling location to the axis of rotation. However, instead of rings that increase in diameter as the test progressed, a spiral shape was formed. Comparing between the decoupled and coupled DoF tests, decoupled pure rotation had better accuracy on holding to the trajectory of the steering guide, only 3.4% away from the desired 71.1 mm radius. These comparisons can be seen between the different laser scanned models in FIG. 26, along with the smooth surface quality.

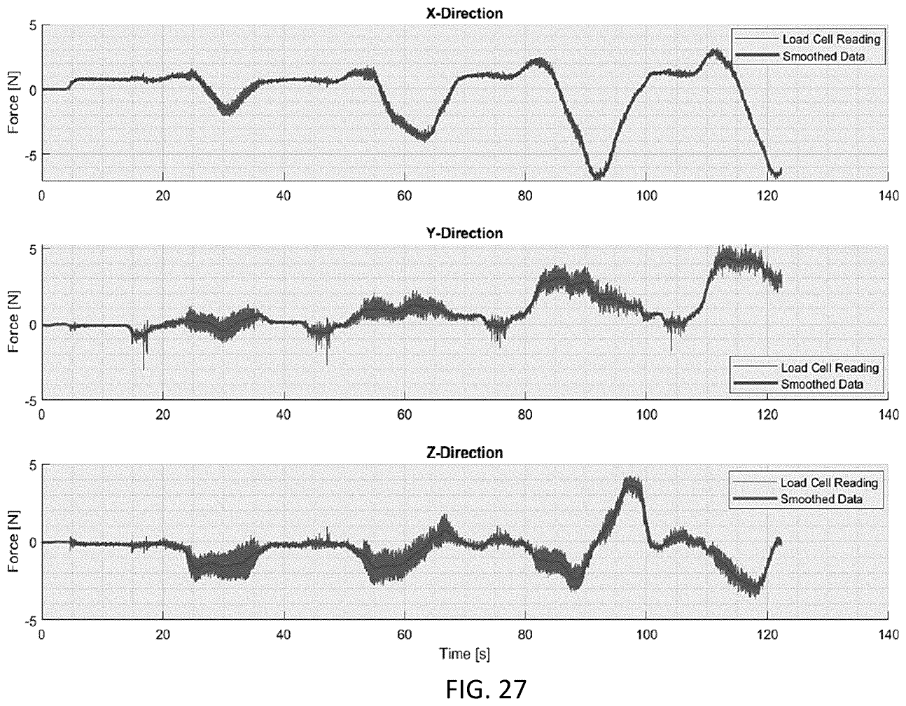

FIG. 27 shows components of both the measured and smooth forces during a pure rotational cavity drilling test performed in 10 PCF Sawbone with the 71.1 mm steering guide. As shown the average directional loads applied to the drill tip and the test sample during a pure rotational cavity drilling experiment using a 10 PCF sawbones block and 71.1 mm radius steering guide. The forces were captured by the load/torque cell (with frequency of 1 kHz) and were smoothed with a span of 100 and averaged in MATLAB using the smooth function (MATLAB, MathWorks). The forces felt in both the X and Z-directions oscillate as the test progresses, as these directions are both perpendicular to the direction of initial cut. As the drill bit cycles around, its primary cutting force is rotated in different directions. The force felt in the Y-direction increased each time the drill was inserted further into the test sample. When the components of the drilling force are resolved into a singular magnitude, the maximum force felt throughout the experiment was 7.13 N.

The system was shown to be capable of reaching up to 82 mm in the perpendicular direction to the point of entry, and surpassing 150° angles with the drill tip for U-shaped path drilling was one of the unique features of the proposed robotic system. Moreover, for the first time, the performance of the system was verified in accurate out-of-the plane J-shape branch and cavity cutting scenarios and performing tests in approximately 2 minutes.

The following references are hereby incorporated herein by reference in their entirety:
O. of the Surgeon General (US et al., “Bone health and osteoporosis: a report of the surgeon general,” 2004.
J. A. Kanis, L. J. Melton III, C. Christiansen, C. C. Johnston, and N. Khaltaev, “The diagnosis of osteoporosis,” Journal of bone and mineral research, vol. 9, no. 8, pp. 1137-1141, 1994.
N. C. Wright, A. C. Looker, K. G. Saag, J. R. Curtis, E. S. Delzell, S. Randall, and B. Dawson-Hughes, “The recent prevalence of osteoporosis and low bone mass in the United States based on bone mineral density at the femoral neck or lumbar spine,” Journal of Bone and Mineral Research, vol. 29, no. 11, pp. 2520-2526, 2014.
R. Burge, B. Dawson-Hughes, D. H. Solomon, J. B. Wong, A. King, and A. Tosteson, “Incidence and economic burden of osteoporosis-related fractures in the United States, 2005-2025,” Journal of bone and mineral research, vol. 22, no. 3, pp. 465-475, 2007.
O. Johnell and J. Kanis, “An estimate of the worldwide prevalence and disability associated with osteoporotic fractures,” Osteoporosis International, vol. 17, no. 12, pp. 1726-1733, 2006.
C. M. Klotzbuecher, P. D. Ross, P. B. Landsman, T. A. Abbott III, and M. Berger, “Patients with prior fractures have an increased risk of future fractures: a summary of the literature and statistical synthesis,” Journal of bone and mineral research, vol. 15, no. 4, pp. 721-739, 2000.
R. Wittenberg, M. Shea, D. Swartz, K. Lee, A. White 3rd, and W. Hayes, “Importance of bone mineral density in instrumented spine fusions.” Spine, vol. 16, no. 6, pp. 647-652, 1991.
K. Okuyama, K. Sato, E. Abe, H. Inaba, Y. Shimada, and H. Murai, “Stability of transpedicle screwing for the osteoporotic spine. an in vitro study of the mechanical stability.” Spine, vol. 18, no. 15, pp. 2240-2245, 1993.
L. Weiser, G. Huber, K. Sellenschloh, L. Viezens, K. P″uschel, M. M. Morlock, and W. Lehmann, “Insufficient stability of pedicle screws in osteoporotic vertebrae: biomechanical correlation of bone mineral density and pedicle screw fixation strength,” European Spine Journal, vol. 26, no. 11, pp. 2891-2897, 2017.
C. L. Goldstein, D. S. Brodke, and T. J. Choma, “Surgical management of spinal conditions in the elderly osteoporotic spine,” Neurosurgery, vol. 77, no. suppl 1, pp. S98-S107, 2015.
F. Alambeigi, M. Bakhtiarinejad, A. Azizi, R. Hegeman, I. lordachita, H. Khanuja, and M. Armand, “Inroads toward robot-assisted internal fixation of bone fractures using a bendable medical screw and the curved drilling technique,” in 2018 7th IEEE International Conference on Biomedical Robotics and Biomechatronics (Biorob). IEEE, 2018, pp. 595-600.
F. Alambeigi, M. Bakhtiarinejad, S. Sefati, R. Hegeman, I. lordachita, H. Khanuja, and M. Armand, “On the use of a continuum manipulator and a bendable medical screw for minimally invasive interventions in orthopedic surgery,” IEEE transactions on medical robotics and bionics, vol. 1, no. 1, pp. 14-21, 2019.
F. Alambeigi, Y. Wang, S. Sefati, C. Gao, R. J. Murphy, I. lordachita, R. H. Taylor, H. Khanuja, and M. Armand, “A curved-drilling approach in core decompression of the femoral head osteonecrosis using a continuum manipulator,” IEEE Robotics and Automation Letters, vol. 2, no. 3, pp. 1480-1487, 2017.
F. Alambeigi, Y. Wang, R. J. Murphy, I. lordachita, and M. Armand, “Toward robot-assisted hard osteolytic lesion treatment using a continuum manipulator,” in 2016 38th Annual International Conference of the IEEE Engineering in Medicine and Biology Society (EMBC). IEEE, 2016, pp. 5103-5106.
Y. Wang, H.-W. Yip, H. Zheng, H. Lin, R. Taylor, and K. W. S. Au, “Design and experimental validation of a miniaturized robotic tendon driven articulated surgical drill for enhancing distal dexterity in minimally invasive spine fusion,” IEEE/ASME Transactions on Mechatronics, 2021.
J. H. Ma, S. Sefati, R. H. Taylor, and M. Armand, “An active steering hand-held robotic system for minimally invasive orthopaedic surgery using a continuum manipulator,” IEEE Robotics and Automation Letters, vol. 6, no. 2, pp. 1622-1629, 2021.
P. E. Dupont, J. Lock, B. Itkowitz, and E. Butler, “Design and control of concentric-tube robots,” IEEE Transactions on Robotics, vol. 26, no. 2, pp. 209-225, 2009.
R. J. Webster III and B. A. Jones, “Design and kinematic modeling of constant curvature continuum robots: A review,” The International Journal of Robotics Research, vol. 29, no. 13, pp. 1661-1683, 2010.
J. Burgner-Kahrs, D. C. Rucker, and H. Choset, “Continuum robots for medical applications: A survey,” IEEE Transactions on Robotics, vol. 31, no. 6, pp. 1261-1280, 2015.
P. E. Dupont, B. J. Nelson, M. Goldfarb, B. Hannaford, A. Menciassi, M. K. O'Malley, N. Simaan, P. Valdastri, and G.-Z. Yang, “A decade retrospective of medical robotics research from 2010 to 2020,” Science Robotics, vol. 6, no. 60, p. eabi8017, 2021.
P. E. Dupont, N. Simaan, H. Choset, and C. Rucker, “Continuum robots for medical interventions,” Proceedings of the IEEE, vol. 110, no. 7, pp. 847-870, 2022.
M. Drexel, G. Selvaduray, and A. Pelton, The effects of cold work and heat treatment on the properties of nitinol wire, 2007, vol. 42665.
D. Hodgson, “Fabrication, heat treatment and joining of nitinol components,” in SMST-2000, Proc. Int. Conf. on Shape Memory and Superelastic Technologies, 2001, pp. 11-24.
A. Cetin and D. A. Bircan, “Experimental investigation of pull-out performance of pedicle screws at different polyurethane (pu) foam densities,” Proceedings of the Institution of Mechanical Engineers, Part H: Journal of Engineering in Medicine, vol. 235, no. 6, pp. 709-716, 2021.
C. Pusceddu, B. Sotgia, R. M. Fele, and L. Melis, “Treatment of bone metastases with microwave thermal ablation,” Journal of Vascular and Interventional Radiology, vol. 24, no. 2, pp. 229-233, 2013.
N. C. Hatrick, J. D. Lucas, A. R. Timothy, and M. A. Smith, “The surgical treatment of metastatic disease of the spine,” Radiotherapy and Oncology, vol. 56, no. 3, pp. 335-339, 2000.
W. B. Jacobs and R. G. Perrin, “Evaluation and treatment of spinal metastases: an overview,” Neurosurgical focus, vol. 11, no. 6, pp. 1-11, 2001.
G. Maccauro, M. S. Spinelli, S. Mauro, C. Perisano, C. Graci, and M. A. Rosa, “Physiopathology of spine metastasis,” International journal of surgical oncology, vol. 2011, 2011.
D. Choi, A. Crockard, C. Bunger, J. Harms, N. Kawahara, C. Mazel, R. Melcher, and K. Tomita, “Review of metastatic spine tumour classification and indications for surgery: the consensus statement of the global spine tumour study group,” European spine journal, vol. 19, no. 2, pp. 215-222, 2010.
T. J. Greenwood, A. Wallace, M. V. Friedman, T. J. Hillen, C. G. Robinson, and J. W. Jennings, “Combined ablation and radiation therapy of spinal metastases: a novel multimodality treatment approach,” Pain physician, vol. 18, no. 6, pp. 573-581, 2015.
M. P. Steinmetz, A. Mekhail, and E. C. Benzel, “Management of metastatic tumors of the spine: strategies and operative indications,” Neurosurgical Focus, vol. 11, no. 6, pp. 1-6, 2001.
P. R. Anchala, W. D. Irving, T. J. Hillen, M. V. Friedman, B. A. Georgy, D. M. Coldwell, N. D. Tran, F. D. Vrionis, A. Brook, and J. W. Jennings, “Treatment of metastatic spinal lesions with a navigational bipolar radiofrequency ablation device: a multicenter retrospective study,” Pain physician, vol. 17, no. 4, 2014.
W. Sze, M. Shelley, I. Held, T. Wilt, and M. Mason, “Palliation of metastatic bone pain: single fraction versus multifraction radiotherapy-a systematic review of randomised trials,” Clinical Oncology, vol. 15, no. 6, pp. 345-352, 2003.
S. Becker, A. Chavanne, R. Spitaler, K. Kropik, N. Aigner, M. Ogon, and H. Redl, “Assessment of different screw augmentation techniques and screw designs in osteoporotic spines,” European Spine Journal, vol. 17, no. 11, pp. 1462-1469, 2008.
D. A. Nussbaum, P. Gailloud, and K. Murphy, “A review of complications associated with vertebroplasty and kyphoplasty as reported to the food and drug administration medical device related web site,” Journal of Vascular and Interventional Radiology, vol. 15, no. 11, pp. 1185-1192, 2004.
S. L. Zuckerman, J. Lim, Y. Yamada, M. H. Bilsky, and I. Laufer, “Brachytherapy in spinal tumors: A systematic review,” World Neurosurgery, vol. 118, pp. e235-e244, 2018. [Online]. Available: https://www.sciencedirect.com/science/article/pii/S1878875018313822
C. L. Brace, “Radiofrequency and microwave ablation of the liver, lung, kidney, and bone: what are the differences?” Current problems in diagnostic radiology, vol. 38, no. 3, pp. 135-143, 2009.
L. B. Kratchman, G. S. Blachon, T. J. Withrow, R. Balachandran, R. F. Labadie, and R. J. Webster, “Design of a bone-attached parallel robot for percutaneous cochlear implantation,” IEEE Transactions on Biomedical Engineering, vol. 58, no. 10, pp. 2904-2910, 2011.
T. Yang, H. B. Da Silva, and L. N. Sekhar, “6-surgical positioning, navigation, important surgical tools, craniotomy, and closure of cranial and spinal wounds,” in Principles of Neurological Surgery (Fourth Edition), fourth edition ed., R. G. Ellenbogen, L. N. Sekhar, N. D. Kitchen, and H. B. da Silva, Eds. Philadelphia: Elsevier, 2018, pp. 103-115.e1.
O. M. Omisore, S. Han, J. Xiong, H. Li, Z. Li, and L. Wang, “A review on flexible robotic systems for minimally invasive surgery,” IEEE Transactions on Systems, Man, and Cybernetics: Systems, vol. 52, no. 1, pp. 631-644, 2022.
Y. Wang, H. Zheng, R. H. Taylor, and K. W. Samuel Au, “A handheld steerable surgical drill with a novel miniaturized articulated joint module for dexterous confined-space bone work,” IEEE Transactions on Biomedical Engineering, vol. 69, no. 9, pp. 2926-2934, 2022.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A drilling robot device, comprising:
an outer tube including a proximal end, a distal end, and a first concentric through hole centered on a longitudinal axis extending from the proximal end to the distal end;
at least one inner tube movably nested within the outer tube including a proximal end, a distal end, and a second concentric through hole centered on the longitudinal axis, wherein the at least one nested inner tube is concentric with the outer tube; and
a flexible drive shaft including a proximal end, a distal end and a tool tip positioned at the distal end, wherein the flexible drive shaft extends through the second concentric through hole of the at least one inner tube, and is configured to provide a rotational torque to the tool tip.

2. The device of claim 1, wherein the flexible drive shaft comprises a torque coil.

3. The device of claim 1, wherein the at least one inner tube is curved.

4. The device of claim 1, wherein the at least one inner tube comprises a curved portion and a linear portion.

5. The device of claim 1, wherein the at least one inner tube is pre-treated to follow a preset curvature.

6. The device of claim 4, wherein the radius of the preset curvature is 5 to 200 mm.

7. The device of claim 1, wherein the at least one inner tube is heat-treated.

8. The device of claim 1, wherein the at least one inner tube is differentially heat-treated.

9. The device of claim 1, wherein the at least one inner tube comprises nitinol.

10. The device of claim 1, wherein the outer tube has a diameter of 1 to 20 mm, a wall thickness of 0.05 to 4 mm, and a length of 5 to 500 mm and the first concentric through hole has a diameter of 1 to 20 mm.

11. The device of claim 1, wherein the at least one inner tube has a diameter of 1 to 20 mm, a wall thickness of 0.05 to 5 mm, and a length of 5 to 500 mm and the second concentric through hole has a diameter of 1 to 20 mm.

12. A drilling robot system, comprising:
a manipulation system configured to provide a manipulative force and a drilling torque; and
a drilling robot device movably connected to the manipulation system and configured to receive the manipulative force, comprising:
an outer tube including a proximal end, a distal end, and a first concentric through hole centered on a longitudinal axis extending from the proximal end to the distal end;
at least one inner tube movably nested within the outer tube including a proximal end, a distal end, and a second concentric through hole centered on the longitudinal axis, wherein the at least one nested inner tube is concentric with the outer tube; and
a flexible drive shaft including a proximal end, a distal end and a tool tip positioned at the distal end, wherein the flexible drive shaft extends through the second concentric through hole of the at least one inner tube, and is configured to provide a rotational torque to the tool tip.

13. The system of claim 12, wherein the manipulation system comprises a handheld manipulator.

14. The system of claim 12, wherein the manipulation system comprises a robotic arm.

15. The system of claim 14, wherein the robotic arm is configured to perform a robotic assisted procedure.

16. The system of claim 15, wherein the robotic assisted procedure comprises a surgical procedure.

17. The system of claim 12, wherein the manipulation system comprises a drill motor configured to provide a torque to the tool tip via the flexible drive shaft, and a hand operated linear slide to translationally actuate the at least one inner tube, flexible drive shaft and tool tip.

18. The system of claim 12, wherein the manipulation system comprises a drill motor configured to provide a torque to the tool tip via the flexible drive shaft, and a translational actuation motor configured to actuate a translational actuation mechanism to translationally actuate the inner tube, flexible drive shaft and tool tip.

19. The system of claim 12, wherein the manipulation system comprises:
a drill spline shaft configured to transfer torque provided by a drill motor to the tool tip via a drill carriage and the flexible drive shaft;
a rotational actuation motor configured to rotate the inner tube via a rotational actuation spline shaft and a main housing unit; and
a translational actuation motor configured to linearly actuate the drilling robot device via rotating a translational actuation lead screw and the main housing unit.

20. A drilling method, comprising:
providing a manipulation system configured to provide a manipulative force and a drilling torque;
providing a concentric tube drilling robot device movably connected to the manipulation system and configured to receive the manipulative force; and
drilling a trajectory via a combination of the manipulative force, drilling torque, and a curvature inherent to the concentric tube drilling robot device.

21. The method of claim 20, wherein the concentric tube drilling robot device comprises:
an outer tube including a proximal end, a distal end, and a first concentric through hole centered on a longitudinal axis extending from the proximal end to the distal end;
at least one inner tube movably nested within the outer tube including a proximal end, a distal end, and a second concentric through hole centered on the longitudinal axis, wherein the at least one nested inner tube is concentric with the outer tube; and
a flexible drive shaft including a proximal end, a distal end and a tool tip positioned at the distal end, wherein the flexible drive shaft extends through the second concentric through hole of the at least one inner tube, and is configured to provide a rotational torque to the tool tip.

22. The method of claim 20, wherein the manipulation system comprises at least one of a handheld manipulator and a robotic arm.

23. The method of claim 20, further comprising characterizing a target bone tissue including identifying regions of osteoporotic bone and bone with low mineral density, and forming the drilling trajectory based on the characterization.

24. The method of claim 23, wherein the drilling trajectory is configured to avoid the identified regions of osteoporotic bone and bone with low mineral density.

25. The method of claim 23, wherein the drilling trajectory is configured to follow a three dimensional curved, long, and complex anatomy in which nerves and vessels need to be avoided during the drilling procedure.

26. The method of claim 23, wherein the step of characterizing the target bone tissue comprises the steps of:
performing one or more quantitative computed tomography (QCT) scans on the target bone tissue;
converting the one or more QCT scans into a three-dimensional finite element model of the target bone tissue; and
demarcating osteoporotic regions or low bone mineral density regions in the three-dimensional finite element model.

27. The method of claim 20, wherein the drilled trajectory comprises at least one of a J-shaped trajectory, an S-shaped trajectory, a U-shaped trajectory, a combination of a linear and a curved trajectory, a multi-segment trajectory, a multiple J-shaped branch trajectory, and a minimally invasive cavity cutting trajectory.
